(19) United States
(12) Patent Application Publication
(10) Pub. No.: US 2014/0154257 A1
(43) Pub. Date: Jun. 5, 2014

(54) Materials and methods for diagnosing and treating asthma and dietary Fru-AGEs related disorders including auto-immune and other diseases found to be associated with elevated RAGE. The specification describes methods to identify and make dietary derived advanced glycation end-products, known as Fru-AGEs, Fru-AGE-haptens, and Fru-AGE immune complexes, and to make monoclonal and polyclonal antibodies to this plurality of bio-molecules for use in immunoassays and for use as therapeutic agents

(71) Applicant: Luanne Robalo DeChristopher, Hopewell Junction, NY (US)

(72) Inventor: Luanne Robalo DeChristopher, Hopewell Junction, NY (US)

(21) Appl. No.: 13/908,750

(22) Filed: Jun. 3, 2013

Related U.S. Application Data
(60) Provisional application No. 61/656,987, filed on Jun. 7, 2012.

(51) Int. Cl. A61K 39/395; G01N 33/543

(52) U.S. Cl. CPC A61K 39/3955; G01N 33/54306
USPC 424/137.1; 436/501; 435/7.92; 506/18; 506/9; 435/7.94; 435/7.93; 435/69.6

(57) ABSTRACT

Present invention is directed to materials and methods useful in diagnosing gut derived advanced glycation end-products (“Fru-AGEs”) associated with asthma, juvenile arthritis and other pro-inflammatory chronic diseases. Fru-AGEs arise from the interaction [fructosylation] between elevated gastro-intestinal excess free fructose and other food/bio-molecules including peptides, proteins, lipids, lipo/glycoproteins and others in the digestive tract. Such Fru-AGEs may further interact with moieties of the systemic circulation of those at risk [fructose malabsorbers], i.e. with acute phase proteins, heat shock proteins, immunoglobulins, esRAGE, and sRAGE. Specification describes methods to identify and/or make such excess-free-fructose derived immunogens, i.e. Fru-AGEs, Fru-AGE-haptens, Fru-AGE immune complexes, and to make monoclonal/polyclonal antibodies to such bio-molecules for use as therapeutic agents and/or for use in immuno-assays including fluorescence enzyme immunoassay, microarray specific immunoglobulin/antibody testing, fluids detection [blood and urine] of GI Fru-AGE/haptens/immune-complexes.

### RELATED U.S. APPLICATION DATA

None

### REFERENCES CITED

### U.S. Patent Documents

U.S. Pat. No. 4,727,036 issued Feb. 23, 1988
U.S. Pat. No. 5,206,144 issued Apr. 27, 1993
U.S. Pat. No. 5,206,144 issued Apr. 27, 1993
U.S. Pat. No. 5,702,704 issued Dec. 30, 1997
U.S. RE39,138 E issued Jun. 20, 2006
U.S. Pat. No. 7,279,295 issued Oct. 9, 2007
U.S. Pat. No. 7,510,687 issued Mar. 31, 2009
2009/011172A1 issued Apr. 30, 2009

### Foreign Patent Documents

EP 1 499 894 B1 in EPO Bulletin 25.02.209 N. 2009 September
ZL 03810029.0 in SIPO PRC Bulletin 08.04.2009
PCT/IT03/00218—WO 03/085401

### Other Publications

Akinbami L. Asthma prevalence, healthcare use and mortality. United States 2003-2005, CDC National Center for Health Statistics [Akinbami]. 2006.
BeMiller J N. 2010. Carbohydrate analysis. In: Nielsen S S, editor. Food analysis, 4th ed., New York: Springer
Beyer P L, Caviar E M, McCallum R W. Fructose intake at current levels in the united states may cause gastrointestinal distress in normal adults. J Am Diet Assoc. 2005; 105:1559-1566. http://www.ncbi.nlm.nih.gov/pubmed/16183355
Cui S W. 2005. Food carbohydrates: chemistry, physical properties, and applications. Boca Raton, Fla.: CRC Press, Taylor & Francis Group.
Bunn H F, Higgins P J. Reaction of monosaccharides with proteins: Possible evolutionary significance. Science. 1981; 213:222-224.
Buckley S T, Ehrhardt C. The receptor for advanced glycation end products (RAGE) and the lung. J Biomed Biotechnol. 2010; 2010:917108.
Cardoso C R, Provinciatto P R, Godoi D F, Vieira T S, Ferreira B R, Teixeira G, Rossi M A, Cunha F Q, Silva J S. B cells are involved in the modulation of pathogenic gut immune response in food-allergic enteropathy. Clin Exp Immunol. 2008; 154:153-161.
Chan W C, White P. Fmoc Solid Phase Peptide Synthesis: A Practical Approach series edited by Chan & White, Oxford University Press, 2000; ISBN-10: 0199637245|ISBN-13: 978-0199637249
Charrier L, Merlin D. The oligopeptide transporter hPepT1: Gateway to the innate immune response. Lab Invest. 2006; 86:538-546.
Colotta F, Allavena P, Sica A, Garlanda C, Mantovani A. Cancer-related inflammation, the seventh hallmark of cancer: Links to genetic instability. Carcinogenesis. 2009; 30:1073-1081
DeFrances C, Cullen K, Kozak L. National hospital discharge survey: 2005 annual summary with detailed diagnosis and procedure data. National Center for Health Statistics [DeFrances]. 2007; Vital Health Statistics 12:165.
Dills W L, Jr. Protein fructosylation: Fructose and the maillard reaction. Am J Clin Nutr. 1993; 58:779 S-787S.
Dupont D, Mandalari G, Molle D, Jardin J, Leonil J, Faulks R M, Wickham M S, Mills E N, Mackie A R. Comparative resistance of food proteins to adult and infant in vitro digestion models. Mol Nutr Food Res. 2010; 54:767-780.
Fields, Solid-Phase Peptide Synthesis, Academic Press, 1997
Foell D, Kucharzik T, Kraft M, Vogl T, Sorg C, Domschke W, Roth J. Neutrophil derived human S100A12 (EN-RAGE) is strongly expressed during chronic active inflammatory bowel disease. Gut. 2003; 52:847-853.
Foell D, Roth J. Proinflammatory S100 proteins in arthritis and autoimmune disease. Arthritis Rheum. 2004; 50:3762-3771.
Geissler S, Hellwig M, Zwarg M, Markwardt F, Henle T, Brandsch M. Transport of the advanced glycation end products alanylpyrraline and pyrralylalanine by the human proton-coupled peptide transporter hPEPT1. J Agric Food Chem. 2010; 58:2543-2547.
Gellisen G. Production of recombinant proteins: novel microbial and eukaryotic expression systems. 2005: ISBN 3527310363.
Gomara R E, Halata M S, Newman L J, Bostwick H E, Berezin S H, Cukaj L, See M C, Medow M S. Fructose intolerance in children presenting with abdominal pain. J Pediatr Gastroenterol Nutr. 2008 September; 47(3):303-8. doi: 10.1097/MPG.0b013e318166cbe4. PubMed PMID: 18728526.
Herold K., Moser B, Chen Y, Zeng S, Yan S F, Ramasamy R, Emond J, Clynes R, Schmidt A M. Receptor for advanced glycation end products (RAGE) in a dash to the rescue: inflammatory signals gone awry in the primal response to stress. J. Leukoc. Biol., 2007, 82, 2, 204-212.
Hilmenyuk T, Bellinghausen I, Heydenreich B, Ilchmann A, Toda M, Grabbe S, Saloga J. Effects of glycation of the model food allergen ovalbumin on antigen uptake and Presentation by human dendritic cells. Immunology. 2010; 129:437-445.
Hoekstra J H, van den Aker J H, Kneepkens C M, Stellaard F, Geypens B, Ghoos Y E. Evaluation of 13CO2 breath tests for the detection of fructose malabsorption. J Lab Clin Med. 1996; 127:303-309.
Hoekstra J H, van Kempen A A, Bijl S B, Kneepkens C M. Fructose breath hydrogen tests. Arch Dis Child. 1993; 68:136-138.
Haley Stephen, Toasa J, Jerardo A. Sugar and Sweeteners Outlook. 2009 Sugar Stocks Projected At Low Levels.www.ers.usda.gov/publications/SSS/2008/SSS253.pdf-2008-09-30
Helliwell P A, Richardson M, Affleck J, Kellett G L. Stimulation of fructose transport across the intestinal brush-border membrane by PMA is mediated by GLUT2 and dynamically regulated by protein kinase C. Biochem J. 2000; 350 Pt 1:149-154.
Hofmann M A, Drury S, Fu C, Qu W, Taguchi A, Lu Y, Avila C, Kambham N, Bierhaus A, Nawroth P, Neurath M F, Slattery T, Beach D, McClary J, Nagashima M, Morser J, Stern D, Schmidt A M. RAGE mediates knovel proinflammatory axis: A central cell surface receptor for S100/calgranulin polypeptides. Cell. 1999; 97:889-901.
Horiuchi S, Araki N, Morino Y. Immunochemical approach to characterize advanced glycation end products of the maillard reaction. evidence for the presence of a common structure. J Biol Chem. 1991; 266:7329-7332.
Howl J. Peptide Synthesis Protocols (Methods in Molecular Biology), Humana Press, 2010; ISBN-10: 1588293173
Jakas A, Katic A, Bionda N, Horvat S. Glycation of a lysine-containing tetrapeptide by D-glucose and D-fructose-influence of different reaction conditions on the formation of Amadori/Heyns products. Carbohydr Res. 2008; 343:2475-2480.
Kellett G L, Brot-Laroche E. Apical GLUT2: A major pathway of intestinal sugar absorption. Diabetes. 2005; 54:3056-3062.
Kellett G L, Helliwell P A. The diffusive component of intestinal glucose absorption is mediated by the glucose-induced recruitment of GLUT2 to the brush-border membrane. Biochem J. 2000; 350 Pt 1:155-162.
Kohler G, Milstein C. Continuous cultures of fused cells secreting antibody of predefined specificity. 1975. Biotechnology. 1992; 24:524-526.
Leturque A, Brot-Laroche E, Le Gall M. GLUT2 mutations, translocation, and receptor function in diet sugar managing. Am J Physiol Endocrinol Metab. 2009; 296:E985-92.
Lin L, Park S, Lakatta E G. RAGE signaling in inflammation and arterial aging. Front Biosci. 2009; 14:1403-1413.
Maleki S J, Chung S Y, Champagne E T, Raufman J P. The effects of roasting on the allergenic properties of peanut proteins. J Allergy Clin Immunol. 2000; 106:763-768.
Morbini P, Villa C, Campo I, Zorzetto M, Inghilleri S, Luisetti M. The receptor for advanced glycation end products and its ligands: A new inflammatory pathway in lung disease? Mod Pathol. 2006; 19:1437-1445.
Matysiak-Budnik T, Moura I C, Arcos-Fajardo M, Lebreton C, Ménard S, Candalh C, Ben-Khalifa K, Dugave C, Tamouza H, van Niel G, Bouhnik Y, Lamarque D, Chaussade S, Malamut G, Cellier C, Cerf-Bensussan N, Monteiro R C, Heyman M. Secretory IgA mediates retrotranscytosis of intact gliadin peptides via the transferrin receptor in celiac disease. J Exp Med. 2008 Jan. 21; 205(1):143-54. doi: 10.1084/jem.20071204. Epub 2007 Dec. 31. PubMed PMID: 18166587; PubMed Central PMCID: PMC2234361.
Milutinovic P S, Alcorn J F, Englert J M, Crum L T, Oury T D. The receptor for advanced glycation end products is a central mediator of asthma pathogenesis. Am J Pathol. 2012 October; 181(4):1215-25. doi: 10.1016/j.ajpath.2012.06.031. Epub 2012 Aug. 11. PubMed PMID: 22889845; PubMed Central PMCID: PMC3463633.
Morcos M, Sayed A A, Bierhaus A, Yard B, Waldherr R, Merz W, Kloeting I, Schleicher E, Mentz S, Abd el Baki R F, Tritschler H, Kasper M, Schwenger V, Hamann A, Dugi K A, Schmidt A M, Stern D, Ziegler R, Haering H U, Andrassy M, van der Woude F, Nawroth P P. Activation of tubular epithelial cells in diabetic nephropathy. Diabetes. 2002; 51:3532-3544.
Park S, Blanck H M, Sherry B, Jones S E, Pan L. Regular-soda intake independent of weight status is associated with asthma among US high school students. J Acad Nutr Diet. 2013 January; 113(1):106-11. doi: 10.1016/j.jand.2012.09.020. PubMed PMID: 23260727.
Oura J, Tamura Y, Kamiguchi K, Kutomi G, Sahara H, Torigoe T, Himi T, Sato N. Extracellular heat shock protein 90 plays a role in translocating chaperoned antigen from endosome to proteasome for generating antigenic peptide to be cross-presented by dendritic cells. Int Immunol. 2011; 23:223-237.
Qian B F, Wahl S M. TGF-beta can leave you breathless. Curr Opin Pharmacol. 2009; 9:454-461.
Ramaswamy K, Malathi P, Caspary W F, Crane R K. Studies on the transport of glucose from disaccharides by hamster small intestine in vitro. II. characteristics of the disaccharidase-related transport system. Biochim Biophys Acta. 1974; 345:39-48.
Riby J E, Fujisawa T, Kretchmer N. Fructose absorption. Am J Clin Nutr. 1993; 58:748 S-753S.
Robalo DeChristopher, 2012, Consumption of Fructose and High Fructose Corn Syrup: Is Fructositis triggered bronchitis, asthma, & auto-immune reactivity merely a side bar in the Etiology of Metabolic Syndrome II (to be defined)?—Evidence and a Hypothesis. New York Medical College Library. 2012
Schmitt E, Gehrmann M, Brunet M, Multhoff G, Garrido C. Intracellular and extracellular functions of heat shock proteins: Repercussions in cancer therapy. J Leukoc Biol. 2007; 81:15-27.
Stewart and Young, Solid Phase Peptide Synthesis, 2d ed., Pierce Chemical Co., 1984 Suarez G, Rajaram R, Oronsky A L, Gawinowicz M A. Nonenzymatic glycation of bovine serum albumin by fructose (fructation). comparison with the maillard reaction initiated by glucose. J Biol Chem. 1989; 264:3674-3679.
Takeuchi M, Iwaki M, Takino J, Shirai H, Kawakami M, Bucala R, Yamagishi S. Immunological detection of fructose-derived advanced glycation end-products. Lab Invest. 2010; 90:1117-1127.
Tam et al., J. Am. Chem. Soc., 105:6442, 1983
Tang S C, Chan L Y, Leung J C, Cheng A S, Lin M, Lan H Y, Lai K N. Differential effects of advanced glycation end-products on renal tubular cell inflammation. Nephrology (Carlton). 2011; 16:417-425.
Urbonaviciute V, Voll R E. High-mobility group box 1 represents a potential marker of disease activity and novel therapeutic target in systemic lupus erythematosus. J Intern Med. 2011; 270:309-318.
van Wijk F, Knippels L. Initiating mechanisms of food allergy: Oral tolerance versus allergic sensitization. Biomed Pharmacother. 2007; 61:8-20.
Ushijima K, Riby J E, Fujisawa T, Kretchmer N. Absorption of fructose by isolated small intestine of rats is via a specific saturable carrier in the absence of glucose and by the disaccharidase-related transport system in the presence of glucose. J Nutr. 1995; 125:2156-2164.
Vaillancourt P. E. Coli Gene Expression Protocols: Methods in Molecular Biology, Series Volume 205. 2011: ISBN13: 9781617373022 and ISBN10: 1617373028.
Vos M B, Kimmons J E, Gillespie C, Welsh J, Blanck H M. Dietary fructose consumption among US children and adults: The third national health and nutrition examination survey. Medscape J Med. 2008; 10:160.
Wasserman D, Hoekstra J H, Tolia V, Taylor C J, Kirschner B S, Takeda J, Bell G I, Taub R, Rand E B. Molecular analysis of the fructose transporter gene (GLUT5) in isolated fructose malabsorption. J Clin Invest. 1996; 98:2398-2402.
Wolf R, Mascia F, Dharamsi A, Howard O M, Cataisson C, Bliskovski V, Winston J, Feigenbaum L, Lichti U, Ruzicka T, Chavakis T, Yuspa S H. Gene from a psoriasis susceptibility locus primes the skin for inflammation. Sci Transl Med. 2010; 2:61ra90.
Wu L, Ma L, Nicholson L F, Black P N. Advanced glycation end products and its receptor (RAGE) are increased in patients with COPD. Respir Med. 2011; 105:329-336.
Xue J, Rai V, Singer D, Chabierski S, Xie J, Reverdatto S, Burz D S, Schmidt A M, Hoffmann R, Shekhtman A. Advanced glycation end product recognition by the receptor for AGEs. Structure. 2011; 19:722-732.
Yan S F, Ramasamy R, Schmidt A M. Soluble RAGE: Therapy and biomarker in unraveling the RAGE axis in chronic disease and aging. Biochem Pharmacol. 2010; 79:1379-1386.
Yilmaz Y, Yonal O, Eren F, Atug O, Hamzaoglu H O. Serum levels of soluble receptor for advanced glycation endproducts (sRAGE) are higher in ulcerative colitis and correlate with disease activity. J Crohns Colitis. 2011; 5:402-406.
Yu L C. Intestinal epithelial barrier dysfunction in food hypersensitivity. J Allergy (Cairo). 2012; 2012:596081.
Zahran H S, Person C J, Bailey C, Moorman J E. “Predictors of Asthma Self-Management Education among Children and Adults—2006-2007 Behavioral Risk Factor Surveillance System Asthma Call-back Survey.” Journal of Asthma 2012; 49(1):98-106.

### DRAWINGS

### Drawing 1

TABLE 1

| Example Number | Sequence illustration | Length |
|---|---|---|
| Example 1 | F (CML/CEL) 1 2 3 | 5 residues |
| Example 2 | F (CML/CEL) 1 2 3 4 5 6 | 8 residues |
| Example 3 | −2 F (CML/CEL) 1 2 3 4 5 | 8 residues |
| Example 4 | −3 −2 F (CML/CEL) 1 2 3 4 | 8 residues |
| Example 5 | F (CML/CEL) 1 2 3 4 5 6 7 8 9 10 | 12 residues |
| Example 6 | −2 F (CML/CEL) 1 2 3 4 5 6 7 8 9 | 12 residues |
| Example 7 | −3 −2 F (CML/CEL) 1 2 3 4 5 6 7 8 | 12 residues |
| Example 8 | −4 −3 −2 F (CML/CEL) 1 2 3 4 5 6 7 | 12 residues |
| Example 9 | −5 −4 −3 −2 F (CML/CEL) 1 2 3 4 5 6 | 12 residues |
| Example 10 | −6 −5 −4 −3 −2 F (CML/CEL) 1 2 3 4 5 | 12 residues |
| Example 11 | −7 −6 −5 −4 −3 −2 F (CML/CEL) 1 2 3 4 | 12 residues |
| Example 12 | −8 −7 −6 −5 −4 −3 −2 F (CML/CEL) 1 2 3 | 12 residues |
| Example 13 | F (CML/CEL) 1 2 3 4 5 6 7 8 9 10 11 12 13 | 15 residues |
| Example 14 | −2 F (CML/CEL) 1 2 3 4 5 6 7 8 9 10 11 12 | 15 residues |
| Example 15 | −3 −2 F (CML/CEL) 1 2 3 4 5 6 7 8 9 10 11 | 15 residues |
| Example 16 | −4 −3 −2 F (CML/CEL) 1 2 3 4 5 6 7 8 9 10 | 15 residues |
| Example 17 | −5 −4 −3 −2 F (CML/CEL) 1 2 3 4 5 6 7 8 9 | 15 residues |
| Example 18 | −6 −5 −4 −3 −2 F (CML/CEL) 1 2 3 4 5 6 7 8 | 15 residues |
| Example 19 | −7 −6 −5 −4 −3 −2 F (CML/CEL) 1 2 3 4 5 6 7 | 15 residues |
| Example 20 | −8 −7 −6 −5 −4 −3 −2 F (CML/CEL) 1 2 3 4 5 6 | 15 residues |
| Example 21 | −9 −8 −7 −6 −5 −4 −3 −2 F (CML/CEL) 1 2 3 4 5 | 15 residues |
| Example 22 | −10 −9 −8 −7 −6 −5 −4 −3 −2 F (CML/CEL) 1 2 3 4 | 15 residues |

In certain embodiments the length of the Fru-AGE may be a minimum of 5 or greater, for example, 5, 6, 7, 8, 9, 10 . . . N residues in length, wherein N may be any number that is a subset of the complete protein, or LP, or GP or other food molecule. For example, this shift to the left is possible only twice for the eight amino acids long Fru-AGE. The shift to the left may continue in any of the other Fru-AGEs for example the 15, or 20, or 25 or 30 residues long sequences until at minimum four amino acids remain to the right of the CEL/CML residue. Table 1 illustrates the number of Fru-AGEs possible for a sequence that satisfies the selection rules herein described, and includes examples for the 5, 8, 12 and 15 amino acids long sequences. The table can of course be extended to include 20, 25, 30 or longer Fru-AGEs sequences, and any lengths in between, with continuous shifts to the left, stopping at the point at which a minimum of four residues remain on the right or carboxy side of the hydrophobic/CEL or CML residues. Phenylalanine (F) is used as example of the hydrophobic residue, but as previously stated, can be any one of the hydrophobic residues previously identified including phenylalanine, isoleucine, tryptophan, leucine, valine, methionine, tyrosine, cysteine, or alanine. The CML/CEL immediately to the right of the hydrophobic residue reflects the fructated residue. It occupies the zero position. The positive numbers reflect right flanking (Carboxy side) residues, and negative numbers reflect left flanking (N-terminus side) residues. Table 1 provides an illustration of the methodology.

### Drawing 2

TABLE 2

| | | CML/CEL containing |
|---|---|---|
| Protein | Species | fragment |
| Beta-lactoglobulin | Bovine | LIVTQTM(CML/CEL)GLDI |
| Beta-lactoglobulin | Bovine | EKT(CML/CEL)IPAVF |
| Beta-lactoglobulin | Bovine | VLDTDY(CML/CEL)KY |
| Beta-lactoglobulin | Bovine | EKFDKAL(CML/CEL)A |
| Beta-lactoglobulin | Bovine | AL(CML/CEL)ALPM |
| Beta-Casein | Bovine | GVSKV(CML/CEL)EAMAPK |
| Ovalbumin | Hen | VLVNAIVF(CML/CEL) |
| | | GLWEKA |
| Ovalbumin | Hen | VYLGA(CML/CEL)DSTR |
| Ovalbumin | Hen | IT(CML/CEL)PNDVYS |
| Ovalbumin | Hen | M(CML/CEL)ILEL |
| Ovalbumin | Hen | I(CML/CEL)VYL |
| Ovalbumin | Hen | ERKI(CML/CEL)VY |
| Ovalbumin | Hen | ESL(CML/CEL)ISQAVHAA |
| | | HAEINE |

One aspect of the present invention is to utilize well characterized biochemical models of gastroduodenal infant, children and adult digestion that are unique to each group in an effort to identify digestion resistant fragments that are more probable for each cohort and thereby good cohort specific fructation candidates. The infant and young children's model differs from the adult model for example with reduced levels of proteases (eightfold reduced for pepsin and tenfold reduced for trypsin and chymotrypsin), phosphatidylcholine and bile salts (Dupont et al., 2009). Fru-AGEs identified via this method are herein referred to as Model Fru-AGEs for use in immunoassays and animal inoculations for antibodies production that are the subjects of the present invention. Table 2 illustrates examples of several Model Fru-AGEs from cow's milk and cheese including beta-lactoglobulin and beta-casein, and hen's egg white protein, ovalubumin, all selected using the methodology for identification, and selection herein described. This table 2 of examples is not intended to be limiting of all Model Fru-AGEs, and serves only as an example. Other such Model Fru-AGEs may arise from other proteins for example in cow's milk and cheese, eggs, oats, wheat, chicken, red meat, and other food categories.

### Drawing 3

TABLE 3

| | | CML/CEL containing |
|---|---|---|
| Protein | Species | fragment |
| Glutenin, high molecular weight | Triticum aestivum | L(CML/CEL)ACQQVMDQQL |
| subunit DX5 | (wheat) | |
| Glutenin, high molecular weight | Triticum aestivum | LL(CML/CEL)RYYPSVTCP |
| subunit DX5 | (wheat) | |
| Glutenin, high molecular weight | Triticum aestivum | L(CML/CEL)VAKAQQLAAQ |
| | (wheat) | |
| Glutenin, high molecular weight | Triticum aestivum | L(CML/CEL)ACQQVMDQQ |
| | (wheat) | |
| Glutenin, high molecular weight | Triticum aestivum | ALL(CML/CEL)RYYPSVTS |
| | (wheat) | |
| Gamma-gliadin | Triticum aestivum | VL(CML/CEL)TLPTMCNVY |
| | (wheat) | |
| avenin | Avena sativa (oat) | M(CML/CEL)LDSCREYVAE |
| avenin | Avena sativa (oat) | W(CML/CEL)WWKGGCEELR |
| avenin | Avena sativa (oat) | WW(CML/CEL)GGCEELRNE |
| avenin | Avena sativa (oat) | L(CML/CEL)IAKSLPTQST |
| avenin | Avena sativa (oat) | L(CML/CEL)NNRGQESGVF |
| avenin | Avena sativa (oat) | TM(CML/CEL)VVAMQTLPA |
| Seed storage globulin1 | Avena sativa (oat) | FL(CML/CEL)PFVSQQGPV |
| Aspartic protease inhibitor 2 | Solanum turberosum | IATV(CML/CEL)LCVSY |
| | (potato) | TIW(CML/CEL)VGNLNAYF |
| Aspartic protease inhibitor 2 | Solanum turberosum | F(CML/CEL)IV(CML/CEL)L |
| | (potato) | SNFGYNLLYCPITPPF |
| Aspartic protease inhibitor 7 | Solanum turberosum | V(CML/CEL)LCGSYTIW(CML/ |
| | (potato) | CEL)VGNINAHLRTMLLETGG |
| Aspartic protease inhibitor 7 | Solanum turberosum | F(CML/CEL)IV(CML/ |
| | (potato) | CEL)SSKFGYNLLYCPLTRH |
| Aspartic protease inhibitor 11 | Solanum turberosum | V(CML/CEL)LCVSYTIW(CML/ |
| | (potato) | CEL)VGNLNAYFRT |
| Aspartic protease inhibitor 11 | Solanum turberosum | F(CML/CEL)IV(CML/ |
| | (potato) | CEL)LSNFGYNLLYCPITPPFL |
| | | CPFCRDDNFCAKVGVVIQN |
| Aspartic protease inhibitor 9 | Solanum turberosum | IW(CML/CEL)VGNLNA |
| | (potato) | YFRTMLLETGGTIG |
| Aspartic protease inhibitor 9 | Solanum turberosum | F(CML/CEL)IVKLSNFGYNL |
| | (potato) | LSCPFTSIICLRCPEDQFCAK |
| Aspartic protease inhibitor 9 | Solanum turberosum | FKIV(CML/CEL)LSNFGYNL |
| | (potato) | LSCPFTSIICLRCPEDQFCAK |
| Aspartic protease inhibitor 10 | Solanum turberosum | W(CML/CEL)VGINAYLRTML |
| | (potato) | LETGGTIGQADSSY |
| Aspartic protease inhibitor 10 | Solanum turberosum | F(CML/CEL)IVKSSIL |
| | (potato) | GYNLLYCPITRPIL |
| Cysteine protease inhibitor 1 | Solanum turberosum | MTVVYI(CML/CEL)FFVKTTKL |
| | (potato) | |
| Cysteine protease inhibitor 1 | Solanum turberosum | DQTVW(CML/CEL)VNDEQLVVT |
| | (potato) | |
| Cysteine protease inhibitor 1 | Solanum turberosum | VGNENDIF(CML/CEL)IMKTDLV |
| | (potato) | |
| Serine protease inhibitor 7 | Solanum turberosum | YTIW(CML/CEL)VGDYDASLG |
| | (potato) | |
| Serine protease inhibitor 7 | Solanum turberosum | SWLIV(CML/CEL)SSQFGYNLL |
| | (potato) | |
| Patatin (potato tuber protein) | Solanum turberosum | GGGI(CML/CEL)GIIPATILEF |
| | (potato) | |
| Patatin (potato tuber protein) | Solanum turberosum | ISSFDI(CML/CEL)TNKPVIFT |
| | (potato) | |
| Patatin (potato tuber protein) | Solanum turberosum | FASI(CML/CEL)SLNYKQMLLL |
| | (potato) | |
| Non-specific lipid-transfer | Zea mays (corn) | AACNCL(CML/CEL)KNAAAGVSG |
| protein - zea m14 | | |
| Actin | Gallus gallus (chicken) | NGSGGLV(CML/CEL)AGFAGDD |
| Actin | Gallus gallus (chicken) | KRGILTL(CML/CEL)YPIEHGI |
| Actin | Gallus gallus (chicken) | DLTDYLM(CML/CEL)ILTERGY |
| Actin | Gallus gallus (chicken) | REIVRDI(CML/CEL)EKLCYVA |
| Actin | Gallus gallus (chicken) | ALAPSTM(CML/CEL)I(CML/ |
| | | CEL)IIAPP |
| Skeletal muscle Tropomyosin beta | Gallus gallus (chicken) | KKKMQML(CML/CEL)LDKENAID |
| Skeletal muscle Tropomyosin beta | Gallus gallus (chicken) | QGLQKKL(CML/CEL)GTEDEV |
| Skeletal muscle Tropomyosin beta | Gallus gallus (chicken) | EKYSESV(CML/CEL)EAQEKL |
| Skeletal muscle Tropomyosin beta | Gallus gallus (chicken) | DESERGM(CML/CEL)VIENRMK |
| Skeletal muscle Tropomyosin beta | Gallus gallus (chicken) | VIENRAM(CML/CEL)DEEKMELQE |
| Skeletal muscle Tropomyosin beta | Gallus gallus (chicken) | VIENRAM(CML/CEL)DEEKME |
| Skeletal muscle Tropomyosin beta | Gallus gallus (chicken) | MELQEMQL(CML/CEL)EAKHI |
| Skeletal muscle Tropomyosin beta | Gallus gallus (chicken) | GDLEEEL(CML/CEL)IVTNNLKSL |
| Skeletal muscle Tropomyosin beta | Gallus gallus (chicken) | KIVTNNL(CML/CEL)SLEAQADKYS |
| Skeletal muscle Tropomyosin beta | Gallus gallus (chicken) | DKYEEEI(CML/CEL)LLGEKL |
| | | (CML/CEL)EAE |
| Skeletal muscle Tropomyosin beta | Gallus gallus (chicken) | EVYAQKM(CML/CEL)YKAISEELDN |
| Skeletal muscle Tropomyosin beta | Gallus gallus (chicken) | Y(CML/CEL)AISEELDNAL |
| Skeletal muscle Tropomyosin alpha | Gallus gallus (chicken) | KKKMQML(CML/CEL)KLDKENAL |
| Skeletal muscle Tropomyosin alpha | Gallus gallus (chicken) | DKYSESL(CML/CEL)KDAQEKLE |
| Skeletal muscle Tropomyosin alpha | Gallus gallus (chicken) | DESERGM(CML/CEL)KVIENRAQ |
| Skeletal muscle Tropomyosin alpha | Gallus gallus (chicken) | EIQEIQL(CML/CEL)KEAKHIAE |
| Skeletal muscle Tropomyosin alpha | Gallus gallus (chicken) | RIMDQTL(CML/CEL)KALMAAED |
| Skeletal muscle Tropomyosin alpha | Gallus gallus (chicken) | DKYEEEI(CML/CEL)KVLTDKLK |
| Skeletal muscle Troponin C | Gallus gallus (chicken) | EEMIAEF(CML/CEL)KAAFDMFDA |
| Skeletal muscle Troponin C | Gallus gallus (chicken) | VMMVRQM(CML/CEL)KEDAKGKS |
| Skeletal muscle Troponin C | Gallus gallus (chicken) | EDIEDLM(CML/CEL)KDSDKNND |
| Skeletal muscle Troponin C | Gallus gallus (chicken) | IDFDEFL(CML/CEL)KMMEGVQ |
| Skeletal muscle Myosin heavy chain | Gallus gallus (chicken) | HPKESFV(CML/CEL)KGTIQSKE |
| Skeletal muscle Myosin heavy chain | Gallus gallus (chicken) | EGGKVTV(CML/CEL)KTEGGETL |
| Skeletal muscle Myosin heavy chain | Gallus gallus (chicken) | GGETLTV(CML/CEL)KEDQVFSM |
| Skeletal muscle Myosin heavy chain | Gallus gallus (chicken) | PAVLYNL(CML/CEL)KERYAAWM |
| Skeletal muscle Myosin heavy chain | Gallus gallus (chicken) | MHYGNL(CML/CEL)FKQKQRE |
| Skeletal muscle Myosin heavy chain | Gallus gallus (chicken) | MHYGNL(CML/CEL)F(CML/ |
| | | CEL)QKQRE |
| Skeletal muscle Myosin heavy chain | Gallus gallus (chicken) | LNSAELL(CML/CEL)ALCYPRV |
| Skeletal muscle Myosin heavy chain | Gallus gallus (chicken) | ALCYPRV(CML/CEL)VGNEFVT |
| Skeletal muscle Myosin heavy chain | Gallus gallus (chicken) | DEKTAIY(CML/CEL)LTGAVMH |
| Skeletal muscle Myosin heavy chain | Gallus gallus (chicken) | KATDTSF(CML/CEL)NKLYDQH |
| Skeletal muscle Myosin heavy chain | Gallus gallus (chicken) | LYQKSSV(CML/CEL)TLALLFA |
| Skeletal muscle Myosin heavy chain | Gallus gallus (chicken) | RVLYADF(CML/CEL)QRYRVLN |
| Skeletal muscle Myosin heavy chain | Gallus gallus (chicken) | GHTKVFF(CML/CEL)AGLLGLL |
| Skeletal muscle Myosin heavy chain | Gallus gallus (chicken) | VRSFMNV(CML/CEL)HWPWMKL |
| Skeletal muscle Myosin heavy chain | Gallus gallus (chicken) | PWMKLFFKI(CML/CEL)PLLKS |
| Skeletal muscle Myosin heavy chain | Gallus gallus (chicken) | FKIKPLL(CML/CEL)SAESEKE |
| Skeletal muscle Myosin heavy chain | Gallus gallus (chicken) | ERCDQLI(CML/CEL)TKIQLEA |
| Skeletal muscle Myosin heavy chain | Gallus gallus (chicken) | RKLEGDL(CML/CEL)LAHDSIM |
| Skeletal muscle Myosin heavy chain | Gallus gallus (chicken) | MQLQKKI(CML/CEL)ELQARIE |
| Skeletal muscle Myosin heavy chain | Gallus gallus (chicken) | EKEKSEL(CML/CEL)MEIDDLA |
| Skeletal muscle Myosin heavy chain | Gallus gallus (chicken) | RHLEEEI(CML/CEL)A(CML/ |
| | | CEL)NALAH |
| Skeletal muscle Myosin heavy chain | Gallus gallus (chicken) | DKILAEW(CML/CEL)QKYEETQ |
| Skeletal muscle Myosin heavy chain | Gallus gallus (chicken) | SLSTELFKM(CML/CEL)NAYEE |
| Skeletal muscle Myosin heavy chain | Gallus gallus (chicken) | QLELNQI(CML/CEL)SEIDRKI |
| Skeletal muscle Myosin heavy chain | Gallus gallus (chicken) | EEDIDQL(CML/CEL)DTQIHLD |
| Skeletal muscle Myosin heavy chain | Gallus gallus (chicken) | EAEQLAL(CML/CEL)GGKKQLQ |
| Skeletal muscle Myosin heavy chain | Gallus gallus (chicken) | KRSAEAV(CML/CEL)GVRKYER |
| Skeletal muscle Myosin heavy chain | Gallus gallus (chicken) | RKYERRV(CML/CEL)ELTYQCE |
| Skeletal muscle Myosin heavy chain | Gallus gallus (chicken) | LVDKLQM(CML/CEL)V(CML/ |
| | | CEL)SYKRQ |

Table 3 provides examples of various length AGEs and Fru-AGEs that are amino acid sequences identified from within food proteins and “selected-for”, using rules of the present invention. The identified fragments contain the requisite lysines preceded by hydrophobic residues (phenylalanine, isoleucine, tryptophan, leucine, valine, methionine, tyrosine, cysteine, or alanine). These sequences are depicted with the post translational CML/CEL modification. Many more lengths are possible for each Fru-AGE containing fragment, wherein the CML/CEL residue that occupies position zero is flanked to the left or right by varying numbers of residues from within the protein sequence as is illustrated in table 1. By following the fragment design rules of the present invention, fragment lengths may be 5, 6, 7, 8, 9, 10, 11, 12, 13, 14, 15, 16, 17, 18, 19, 20, 21, 22, 23, 24, 25, 26, 27, 28, 29, 30 or longer.

### BACKGROUND OF THE INVENTION

### Axis of Food Allergy and Asthma

According to the US Center for Disease Control (CDC), the prevalence and number of persons with asthma continues to increase to unprecedented levels, and demographic differences among population subgroups persist despite significant improvements in outdoor air quality [thanks to the Clean Air Act], and despite decreases in cigarette smoking and secondhand smoke exposure (Zahran et al., 2011). These sharp increases in asthma have confounded researchers, and while many hypotheses have been offered, insights to explain the rising trend remain elusive. What remains certain is that other—unknown factors appear to be driving up these rates. The December 2010 Guidelines for the Diagnosis and Management of Food Allergy in the United States (the Guidelines) authored by an expert panel, published by the National Institute of Allergy and Infectious Disease (NIAID) state that food allergy may coexist with asthma associated disorders and eosinophilic esophagitis (EoE), and that in patients with asthma, the coexistence of food allergy may be a risk factor for severe asthma exacerbations. This link and mechanisms that relate the two are not well understood. While the link between aero-allergens and asthma has been studied extensively and is well characterized in the scientific literature, the same is not the case with the association between food immunogens and asthma.

### The Fructositis Hypothesis

As described in a 2012 report by Robalo DeChristopher titled, “Consumption of Fructose and High Fructose Corn Syrup: Is Fructositis triggered bronchitis, asthma, & auto-immune reactivity merely a side bar in the Etiology of Metabolic Syndrome II (to be defined)?—Evidence and a Hypothesis”, the contribution that intestinally derived advanced glycation end products (AGEs) may play in the axis between food “allergy” and asthma is understudied, and tools that measure them and the effects they may exert in pro-inflammatory respiratory symptomology and asthma are not available. The proposed mechanism that relates food allergy and asthma is consistent with the mechanistic flow of ingested or intestinally fructated (fructosylated) peptides and peptide containing food fragments that are able to cross the intestinal epithelial barrier and gain access to the systemic circulation. In the proposed model, such Fru-AGE immunogens travel through the lymphatics to be drained via the thoracic duct to the systemic (blood) circulation, thence continue to the superior vena cava, right atrium, right ventricle, and onto to the lungs.

### Fru-AGE, RAGE and the Lungs

The lungs are the human tissue known to have the highest concentrations of receptors known to bind advanced glycation end-products (RAGE) (Buckley et al., 2010). There is definitive proof that RAGE binds N-epsilon-(carboxy-ethyl)lysine (CEL) or N-epsilon-(carboxy-methyl)lysine (CML) containing peptides as small as seven amino acids in length, that are capable of oligomerization and pro-inflammatory signal transduction (Xue, et al., 2011). These moieties, end products of fructosylation and glycosylation have been well studied in the context of diabetes related pathology and overlooked with respect to the potential for their formation in the gastrointestinal tract during conditions of elevated dietary fructose and high fructose corn syrup consumption.

In 2006, Morbini et al., reported higher levels of RAGE (relative to normal conditions) are expressed in abnormal conditions in bronchial epithelia, type II pneumocytes, endothelia and macrophages and that RAGE over-expression occurs in all pathological conditions associated with RAGE ligand binding and may have a role in chronic lung diseases. In their findings they suggest that the activation of the inflammatory pathway controlled by RAGE is not specific of a single lung disease, however, it may be relevant as a nonspecific pathway of sustained inflammation in lung tissue (Morbini et al., 2006). Recent advances in food allergy associated asthma research suggest that the receptor of advanced glycation end products (RAGE) is central to the axis of food allergy and asthma. Using murine RAGE knockouts, scientists showed that the absence of RAGE abolished most assessed measures of [lung] pathology, including airway hypersensitivity, eosinophilic inflammation, and airway remodeling and that RAGE is a central mediator of asthma pathogenesis (Milutinovic et al, 2012).

### Diagnostic and Therapeutic Tools of the Present Invention—Beyond the Lungs

The present invention relates to materials and methods, in particular the provision of a methodology for identification, determination, selection, and design for use in synthesis of such target moieties suitable for molecular allergy diagnosis by for example, fluorescence enzyme immunoassay (FEIA); microarray based specific antibody testing including for example immunoglobulin/IgG/IgE/IgA; and antibody production (polyclonal and monoclonal) for therapeutic purposes and symptoms management; and for ELISA based or other testing to be used in diagnosis of pro-inflammatory respiratory disease states associated with fructositis; and as may be relevant to other RAGE related pro-inflammatory, auto-immune diseases, heart disease, atherosclerosis and cancer. Materials include post-translationally modified dietary proteins and peptides (P), lipoproteins/lipopeptides (LPs) or glycoproteins/glycopeptides (GPs), Fru-AGE-haptens, and Fru-AGE-immune complexes—all herein referred to as—all Fru-AGEs; reagents and kits for fluorescence enzyme immunoassay and multiplexed microarray specific immunoglobulin/IgE/IgG testing; and utilization of all Fru-AGEs in monoclonal and polyclonal antibodies production useful as therapeutic agents, and/or for detection and measurement of such immunogens in body fluids including serum, blood and urine. In immuno-assays such antibodies would be useful in assessing levels of post-translationally modified dietary peptides, proteins, LPs, GPs, Fru-AGE-haptens as may be formed in the systemic circulation of susceptible individuals; and Fru-AGE immune complexes as may occur when any of these Fru-AGE bio-molecules interacts with and binds immuno-regulatory proteins, including, but not limited to immunoglobulins, esRAGE, and sRAGE of the immune system.

These diagnostic and therapeutic tools and methods may be useful in assessing the role of dietary and intestinally fructated (fructosylated) immunogens capable of eliciting an adverse immune response, of binding receptors of advanced glycation end products (RAGE) and triggering a pro-inflammatory signal transduction cascade, immune reactivity, and symptoms consistent with aforementioned fructositis disease including: cough not associated with a cold or flu; fever; mucus hypersecretion; inflamed and enlarged tonsils; chronic bronchitis; bronchitis triggered asthma; Eustachian tube dysfunction; otitis media (inner ear infections); atypical pneumonitis; and aberrant respiratory symptomology, and in other associated pro-inflammatory disease states associated with elevated RAGE. Use of monoclonal and polyclonal antibodies of the present invention as therapeutic agents may be relevant in symptoms management and treatment of fructositis disease and other pro-inflammatory, RAGE associated disease states including neutrophilic asthma, COPD; arthritis; rheumatoid arthritis; Systemic Lupus Erthematosus (SLE); Alzheimer's; atherosclerosis; heart disease; peripheral vascular disease (PVD); diabetic retinopathy, neurophathy, and nephropathy; lung cancer and fibrosis; irritable bowel disease; ulcerative colitis; Chron's disease; psoriasis; cancer; and others.

The present invention may also be useful in assessing the levels of such immunogens and presence of related antibodies as may be contributing to pro-inflammatory states associated with other auto-immune diseases wherein RAGE has been linked and elevated. In addition to measuring dietary Fru-AGE levels relative to a standard, in order to assess the role of fructose malabsorption/fructose intolerance associated immunogens formation and their role in RAGE triggered respiratory symptomology, the diagnostic tools that are the subject of the present invention may be useful in assessing the role that such dietary Fru-AGE immunogens may exert in diseases associated with immune hypersensitivity and RAGE signaling including Systemic Lupus Erythematosus, also known as SLE or “Lupus” (Urbonaviciute and Voll, 2011); Alzheimer's disease (M. A. Hofmann et al., 1999; Lin et al., 2009; Yan et al., 2010), atherosclerosis, heart disease, and congestive heart failure; peripheral vascular diseases, also known as PVD (Lin et al., 2009; Yan et al., 2010); diabetic retinopathy, neurophathy, and nephropathy (Lin et al., 2009; Yan et al., 2010); arthritis and rheumatoid arthritis, also known as RA (Foell and Roth, 2004; Lin et al., 2009; Yan et al., 2010); pulmonary disorders (Morbini et al., 2006) including neutrophilic asthma and COPD (Lin et al., 2009; Wu, Ma, Nicholson, and Black, 2011); lung cancer and fibrosis (Buckley and Ehrhardt, 2010); Irritable Bowel Disease (Syndrome), also known IBD (Foell et al., 2003); ulcerative colitis and Crohn's (Yilmaz, Yonal, Eren, Atug, and Hamzaoglu, 2011); Psoriasis (Wolf et al., 2010).

Future research must explore the potential role and possible biochemical linkage between fructose intolerance, fructose malabsorption, high levels of dietary fructose, high fructose corn syrup (HFCS), Fru-AGE, RAGE signaling and these diseases. What appears clear is that to the extent that AGEs may beget further AGE formation and AGE interaction with RAGE increases oxidative stress signals (Herold et al., 2007), any further insult from dietary and intestinally derived AGEs (as may occur in fructose malabsorbers and fructose intolerance sufferers) would only exacerbate any underlying disease conditions. Given the plurality of pro-inflammatory and RAGE related diseases wherein constitutive RAGE signaling, and elevations in pro-inflammatory NF-kappaB and IL-6 are linked, the present invention may have diagnostic value that extends beyond lung related respiratory symptomology, asthma, atypical pneumonitis, and COPD. For example, it may be of value and offer a more complete assessment of risk factors for diseases wherein inflammation is an underlying theme including autoimmune disorders, atherosclerosis, heart disease, diabetes and cancer.

### Diagnostic and Therapeutic Tools of the Present Invention—Cancer

As relates to cancer, it has been well documented that in the cancer extrinsic pathway, inflammatory conditions facilitate cancer development. The triggers of chronic inflammation that increase cancer risk or progression include infections, auto-immune diseases, and inflammatory conditions of uncertain origin. In addition to these cancer associated risk factors, numerous transcription factors and pro-inflammatory cytokines have been identified as prime movers of cancer related inflammation, including the nuclear transcription factor NF-kB and cytokines IL-1B, IL-6 and TNF-alpha (Colotta et., al 2009). This knowledge, combined with the numerous studies that have confirmed that RAGE when bound and oligomerized precipitates a pro-inflammatory signal transduction cascade resulting in elevations of NF-kappaB and IL-6 (Xue et al., 2011), suggests that intersections and compensatory pathways may exist between a Fru-AGE/RAGE/NF-kappaB/IL-6 pathway and underlying chronic inflammation that increases cancer risk and progression. Using a house dust mite mouse model of asthma/allergic airway disease, researchers found that the absence of RAGE abolishes most assessed measures of pathology, including airway hypersensitivity, eosinophilic inflammation, and airway remodeling (Milutinovic et al., 2012).

### The Axis of Dietary Fru-AGEs, RAGE, Cytokines and the Lungs

Researchers across several independent groups (Hilmenyuk et al., 2010; Morcos et al., 2002; Tang et al., 2011), have found that IL-6 becomes highly expressed upon activation of the AGE/RAGE/MAPK-1/NF-kappaB pathway. This finding is significant when considered in the context of recent research by the US National Institute of Health (NIH) relating to lung disease. According to researchers Qian and Wahl of the National Institutes of Health, what has emerged is in fact far more complex than the T helper 0 (Th0) to Th1/Th2 classical model of T helper cell differentiation. For example, beyond the role of TGF-beta in lung homeostasis, there exists an interplay between TGF-beta and IL-6 in inflammatory conditions that leads to goblet cell hyperplasia, mucus hypersecretion, and bronchial hyper-responsiveness, tissue damage, aberrant repair, airway constriction, obstruction, and remodeling (Qian and Wahl, 2009). These works suggest a compensatory pathway may exist between the Fru-AGE/RAGE/NF-kappaB/IL-6 pathway and lung related pathologies.

In their proposed model, Naïve CD4+ T lymphocytes are largely influenced by TGF-beta which has been shown to be indispensable for the development of Th17 populations and Treg's. Both appear to have critical functions in the evolution of inflammation and in the resolution of the response. In their 2009 report titled, “TGF-beta can leave you breathless”, Qian and Wahl provide a new lung model of airway inflammation that incorporates what has been learned recently about the role of TGF-beta with IL-6 in helper T cell differentiation and maturation. What has emerged is a complex pathway including Th0 differentiation into Treg, Th17, and/or Th2, and/or Th9 cells all mediated by release of numerous cytokines including IL-17, IL-4, IL13, IL5, IL9 and others that orchestrate the activation and suppression profiles of cells within the environment and leading to the aforementioned symptoms, which can eventuate in compromised lung function and lung pathology (Qian and Wahl, 2009).

Dietary Fru-AGEs—Considerations in Absorption, and Transport Food allergens are known to cross the epithelial barrier and gain access to the systemic circulation wherein they trigger allergic symptoms in susceptible individuals. Many mechanisms and routes of allergen entry have been identified including transport via the PepT1 transporter (Geissler et al., 2010; Charrier and Merlin, 2006), transcellulary through the Peyer's patch M cells (van Wijk et al., 2006), trans and paracellularly through the intestinal epithelium (Yu, 2012), and mediated via CD23/low affinity IgE (Yu, 2012) and CD71/IgA (Matysiak-Budnik, 2008). In one such example, using allergic animal models, accumulating evidence suggests that a low-affinity IgE receptor (CD23/FcεRII) may contribute to enhanced antigen recognition and rapid transepithelial transport. These data support the notion that food allergens binding to IgE/CD23 are protected from lysosomal degradation in intestinal epithelium, and therefore, intact antigenic forms of the proteins are preserved during transcytosis. It is now clear that IgE/CD23 plays a major role in the mechanism of enhanced transepithelial antigen uptake that is responsible for later mast cell activation and anaphylactic responses in experimental models. CD23 was previously known for its role in regulating IgE synthesis in B cells and promoting B cell proliferation. CD23 expression was found in small intestinal epithelial cells in normal and food-allergic humans and rodents, as well as in bronchial epithelial cells in asthmatic patients. The phenomenon of increased antigen uptake within the endosomal compartment was observed in jejunal enterocytes before the occurrence of mast cell activation, suggesting that heightened apical-to-basolateral transcellular transport of allergen is mast cell independent. Also during the period following mast cell activation, antigens were visualized not only inside endosomes but also within the tight junctions and paracellular regions between enterocytes in allergic animals, suggesting a crucial role of mast cell activation in the induction of tight junction opening and increase of paracellular influx that was not antigen specific. (Yu, 2012). Additionally, protease resistant peptides as large as 33-mer have been shown to transport across the epithelium in celiac disease. Work by (Matysiak-Budnik, 2008) elucidates how transferrin receptor CD71 is responsible for apical to basal retrotranscytosis of gliadin peptides, a process during which p31-49 and 33-mer peptides are protected from degradation.

It is now well recognized that responses to orally administered antigens have both local and systemic features that distinguish them from other immunological reactions (Cardoso et al., 2008). Breakdown of mucosal tolerance to fed antigens can lead to the development of enteropathies that trigger immune cell responses that are observed beyond the confines of the digestive tract and often involve IgE producing B cells in response to activated T-helper 2 (Th2) effector lymphocytes and cytokines (Cardoso et al., 2008).

### The Unexplained Rise in Asthma, Parallels Increases in HFCS Intake, Pivots Upward in 1980

In 2007, 29% of children who had a food allergy also had asthma (Akinbami, 2006), yet the mechanisms that relate the two continue to confound researchers. Given our improvements in air quality and lower indoor smoking rates, asthma rates should be declining—not rising. According to the Asthma and Allergy Foundation of America (AAFA), several studies indicate that during the years from 1980 to 1994, asthma increased among school children by 80 percent and among preschool children by 160 percent. In 2005, 8.9% of children in the United states had asthma. A November 2007 report from the American Lung Association titled, Trends in Asthma Morbidity and Mortality, cites that an average of one of every 10 school-aged children had asthma. This trend has affected black children much more significantly than children of other races and ethnicities. In 2009, according to a May 2011 Vital Signs Report from the Centers for Disease Control (CDC) and Prevention, about 1 in 6 (17%) of non-hispanic black children had asthma, the highest rate among racial/ethnic groups. These dramatic increases are not without consequences as asthma is the third-ranking cause of hospitalization among children under 15 (DeFrances, Cullen, and Kozak, 2007). According to the National Heart, Lung and Blood Institute of the U.S. National Institute of Health, estimated 1998 direct and indirect monetary costs for asthma totaled $11.3 billion. Asthma related medical costs continue to climb. Two decades later, 2007 CDC estimates are that asthma costs the US approximately $56 billion in medical costs, lost school and work days, and early deaths.

The dramatic rise in asthma amongst children during the period from 1980 to 1994 coincides with another trend occurring during this same time frame, a parallel trend not usually discussed in the context of rising rates of asthma. According to the United States Department of Agriculture's (USDA) September 2008 report by Haley et al titled, Sugar and Sweeteners Outlook, the early 1980's also coincides with when the US food industry began a steady increase in their use of high fructose corn syrup (HFCS) as the preferred sweetener (Haley, Toasa, and Jerardo, 2008). According to the USDA, since 1985, the rise in sugar demand, although strong, has been moderate compared with the growth of corn sweeteners. In 1980, per capita consumption of HFCS was approximately 27 pounds; by 1999 it had grown to 60.3 pounds. Thus by 1999 per capita consumption had more than doubled from its 1980 levels. By 1999 Americans were consuming more than a pound per week of HFCS.

Given that presently available diagnostic tools are often inadequate in identifying the source of food allergy attributed asthma and related symptoms, and do not assess, measure nor address the role that dietary Fru-AGEs may exert in RAGE related inflammation and asthma—tools that aid in the measurement and assessment of all (dietary) Fru-AGEs in body fluids including serum, blood and urine samples, of all (dietary) Fru-AGEs antibodies and titers relative to established standards, and of (dietary) Fru-AGE-immune complexes would be desirable. These are unavailable today. Studies, methods and technologies used to date to measure food allergens are specific for native and recombinant allergens, and do not address food allergens that arise from post-translational modifications as described herein. More than 170 foods have been reported to cause IgE-mediated reactions. The focus in development of diagnostic tools and methods has largely targeted the most common foods including peanut, tree nuts, crustacean shellfish, cow's milk, hen's egg, wheat and soy. Since native food proteins have garnered all of the attention in food allergy diagnosis and management, the role that sugars, and in particular the role that fructose might play in modifying dietary proteins capable of eliciting an allergy response has been unaddressed. Consistent with this lack of general awareness is a lag in development of studies, methods and technologies useful in diagnosis of individuals with such sensitivities. In those at risk, particularly fructose malabsorbers, and fructose intolerance sufferers the root cause of allergy symptoms remains elusive. This is complicated by the fact that medical professionals indicate that fructose malabsorption and fructose intolerance may be under-recognized and under-diagnosed, particularly in children (Gomara, et al., 2008).

### The Symptoms

Current methods and technologies used for food allergy diagnosis are of no value in detection of novel post-translationally modified allergens as may occur through food processing or form in the gastrointestinal (GI) tract of susceptible individuals, or further react to form larger pro-inflammatory complexes in the systemic circulation of those at risk. Once these novel allergens gain access to the systemic circulation, they may induce aberrant symptoms consistent with fructositis including atypical pneumonitis, aberrant respiratory symptomology, and asthma. Aside from an early onset dry cough, that is not associated with a cold or flu (and often lasts through the night), symptoms do not follow the phenotype most often associated with food borne allergies. The allergic reaction as described in the case report by Robalo DeChristopher, 2012, is not immediate as often occurs in anaphylaxis, rather, it is somewhat delayed and begins with a dry hacking type cough, followed by low grade fever, bronchial mucus hypersecretion, and airway hyper-reactivity. Abdominal gas and bloating maybe experienced concurrently with early symptoms. If unresolved and mucus levels remain elevated, the symptoms often progress within days to bronchial infection, bronchitis triggered asthma, chronic bronchitis, wheezing, whistling, rattling, allergic rhinitis, otitis media (inner ear infections), and Eustachian tube dysfunction (Robalo DeChristopher, 2012). Therefore, diagnostic tools that effectively measure biomarkers of fructositis disease would be useful and relevant in the development and assessment of food allergy management strategies that could help such individuals. Of particular benefit would be a diagnostic tool that is non-invasive, especially as it relates to young children, that is, one that can be easily used to detect and measure the presence of such immunogens for example in urine.

### Sugar Chemistry—how Effects of pH, Temperature and High Fructose Set the Stage for the Perfect Storm

The reaction of sugars are heavily influenced by pH and temperature. When a reducing sugar is dissolved in water [fructose in this case], hydrolysis, and ring opening and subsequent ring closure occurs, thereby producing the [symbol] - and β-pyranose and [symbol] - and β-furanose ring forms of fructose. These forms have different chemical and physical properties (e.g., optical rotation, solubility, chemical reactivity, relative sweetness, etc.). At a given pH and temperature, the conformation of fructose exists in an equilibrium between these four different ring forms [referred to as tautomers] and the reactive open chain form (BeMiller, 2010; Cui et al, 2005; Suarez et al, 1989). At higher temperatures [ie body temperature] more of fructose exists in the higher energy [symbol] -pyranose, [symbol] -furanose forms and larger amounts are in open-chain form (Suarez et al, 1989). This is significant in the context of Fru-AGEs formation in the GI because in the open chain form more of the partially positive carbonyl carbons are exposed and capable of reacting with dietary proteins in the well-studied Maillard reaction. The Maillard reaction occurs at a more rapid rate proportional to the amount of sugar in the open-chain from. Pentose sugars (fructose) have higher amounts in the open-chain form than hexose sugars (glucose). For example, at 31° C. (87.8° F.) 0.8 percent of fructose is in open chain form. This is why glucose is considered less reactive [than fructose] because at 31° C. (87.8° F.) less of it (0.002) is in open-chain form (Cui, 2005). This means that 400 times more fructose is in the reactive open-chain form than is glucose. See the following table as adapted from Cui et al 2005.

Distribution of D-Sugar Tautomers
at Equilibrium in Aqueous Solution

| | | α- | β- | α- | β- | Open |
|---|---|---|---|---|---|---|
| | Temp | Pyranose | Pyranose | Furanose | Furanose | chain |
| Sugar | ° C. | % | % | % | % | Type % |
| fructose | 31 | 2.5 | 65 | 6.5 | 25 | 0.800 |
| glucose | 31 | 38 | 62 | 0.5 | 0.5 | 0.002 |

Adapted from Cui 2005.

A change in pH, and/or temperature can shift fructose's anomeric equilibrium to it's more reactive open chain form. For example, increased acid and/or increased base conditions, ie movements in either direction away from neutral pH, will shift the equilibrium to more of the [reactive] open chain form. The equilibrium of fructose shifts more to the reactive open chain form in acid as well as base conditions because ring opening can be catalyzed by both acid and base. In acid catalysis, attack by a proton (acid) on the ring oxygen of fructose results in a change in equilibrium between each of the two epimers (furanose and pyranose) and in increased ring opening. Under alkaline conditions hydroxide ion (base) initiates proton removal from the anomeric hydroxyl of fructose, followed by ring opening (BeMiller, 2010, Dills et al, 1993, Suarez et al, 1989).

These are important considerations in the potential for Fru-AGEs formation in the intestines of fructose malabsorbers. The effects of increased concentrations of unabsorbed fructose must be considered in the context of body temperature, in conjunction with changes in pH at different points of the gastrointestinal tract. For example, body temperature will shift fructose contained in a cold soda to the more of the open chain form, as will the lower pH of the stomach (pH 2-3), as will the elevated pH of the duodenum and upper ileum (pH 9.X). These conditions all increase the potential for reactivity and dietary Fru-AGEs formation via the non-enzymatic Maillard reaction.

### Isomeric Forms of Fructose

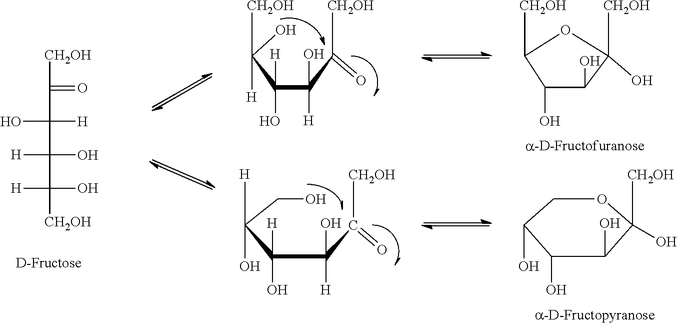

The Chemical Reactivity of Fructose with [Dietary] Proteins—Applying What we Know

As previously noted, the Maillard reaction and its health consequences have been well studied in the context of diabetes and diabetes related pathologies. Reducing sugars such as fructose and glucose are known to react with the amino group in proteins in a non-enzymatic reaction known as the Maillard reaction, creating intermediates known as Amadori and Heyns rearrangements, and flourescent and cross-linked end products known as advanced glycation end products (AGE) and more specifically Fru-AGE when the reducing sugar is fructose. In these reactions, the sugar's carbonyl group chemically reacts with the nucleophilic amino groups of amino acids arginine and to a greater extent lysine. Intermediates known as Amadori and Heyns rearrangements are formed (Horiuchi, Araki, and Morino, 1991; Takeuchi et al., 2010). These intermediates of the reaction go on to form CML and CEL capable of binding the receptor for advanced glycation end products (RAGE) as elucidated in works by Xue et al., May 2011, wherein the structure of the receptor for AGEs (RAGE) bound to CEL modified peptides was determined by NMR spectroscopy. This research provided definitive proof that post-translationally modified peptide fragments as small as seven amino acids in length bind RAGE with much greater affinity than native peptide controls, and that they are capable of oligomerization induced signal transduction resulting in pro-inflammatory cytokines gene expression (Xue et al., 2011).

Research conducted in 2000 by the US Department of Agriculture is consistent with these findings. Their work does not involve RAGE per se. Rather, the intersection between these studies is in the fact that Maillard reaction products, e.g. post-translationally modified molecules containing CML and CEL epitopes are directly involved in activation of the immune system. USDA research by Maleki et. al., points to the formation of novel allergens in peanut proteins that have undergone the Maillard reaction. They confirmed increased access of large immunogens across the intestinal barrier by way of cross-linking and multimer formation of peanut protein antigens that have undergone the Maillard reaction. Ara h 1 is cross-linked through the Maillard reaction to form covalently associated trimers and hexamers. Trimeric complex formation may afford the molecule some protection from protease digestion and denaturation. This work showed passage of large fragments of Ara h 1 that contain several intact IgE-binding sites, across the lumen of the small intestine and demonstrated how the Maillard reaction gives rise to new epitopes. Maleki et al., sought to assess the biochemical effects of roasting and the Maillard reaction on the allergenic properties of peanut proteins. They observed that roasted peanuts bound IgE from patients with peanut allergy at approximately 90-fold higher levels than the raw peanuts from the same cultivars. To determine the contribution of the Maillard reaction to the increase in allergenic properties of the major peanut allergens Ara h 1 and Ara h 2, they subjected these two strains to the Maillard reaction using an in vitro model. The authors report that these known peanut allergens bound higher levels of IgE and were more resistant to heat and digestion by gastrointestinal enzymes once they had undergone the Maillard reaction, but that the Maillard reaction increase in IgE binding was not enough to account for the larger increase seen in IgE binding by roasted peanut extracts. There are also differences in IgE binding sites after peanut antigens for example Ara h 1, Ara h 2 are subjected to roasting or modified by the Maillard reaction (Maleki et al., 2000).

This is significant in that it confirms the formation of new epitopes occurring in food that has undergone the Maillard reaction. Therefore, it is possible that individuals with no known allergy to raw peanuts may in fact be allergic to post-translationally modified peanuts proteins that have undergone the Maillard reaction. Such individuals may have immune cells capable of binding the novel epitopes and triggering an immune response. In these cases, existing diagnostic methods would be of no use in detection and measurement of such novel allergens and in the diagnosis of individuals who have no immune response to the native proteins but do have an aberrant immune response to the newly formed, novel antigens.

### Fructose Malabsorption—the Inconvenient Truth—Understudied & Under-Diagnosed

A review of research regarding rates of fructose malabsorption, and research relating to transport of fructose versus sucrose across the intestinal barrier is critical to understanding the role that elevated dietary fructose and HFCS may have in intestinal formation of novel post-translationally modified immunogens and the percentages of the population that may be affected. Research indicates that large percentages of fructose consumers are fructose malabsorbers. Scant research has been done in children. What is available suggests children are at higher risk of being fructose malabsorbers (Riby, et al., 1993; Gomara et al., 2008). Research conducted during the 1980's and 1990's (Riby et al., 1993), and again in 2005 (Beyer et al., 2005) by various research groups indicates that 10% to 30% or more of “healthy” adults are fructose malabsorbers when consuming fructose in excess of glucose at rates reflective of the western diet (70 grams-100 grams daily) (Vos et al., 2008). Malabsorption rates were observed with consumption of increasing ratios and concentrations from 2:1 (50 g/25 g) to 4:1 (50 g/12.5 g) of fructose, and glucose respectively. Even at levels as low as 12 grams daily consumption of fructose, 10% of study subjects were found to be fructose malabsorbers (Riby et al., 1993). Very little research has been done in children and the little that has been done indicates high rates of malabsorption in children. For example, in 2008, researchers at New York Medical College assessed levels of fructose malabsorption/fructose intolerance in children presenting with abdominal pain and found 30% of those tested were positive for fructose intolerance and that the disease may be under-diagnosed (Gomara et al., 2008). Researchers in another study found that when challenged with 1 gram of fructose per kg of weight, 25 of 57 (44%) children ages 0.1 to 6 years showed incomplete absorption. Of this group, the percentage incompletely absorbing fructose and the peak breath hydrogen value were significantly higher in children aged 1-3 years (Hoekstra et al., 1993).

### Differences in Glucose, Fructose and Excess-Free-Fructose Transport—why it Matters

A review of research into the causes of fructose malabsorption/fructose intolerance elucidates the different mechanisms used for transport of fructose monomers versus the digestion products of sucrose, and is relevant in providing insights into how fructose malabsorption may occur and how elevated levels may contribute to Fru-AGEs formation and symptoms of fructositis. It is also relevant to understanding how consumption of elevated dietary fructose and HFCS, made of unbound monomers of fructose and glucose, can trigger an immune response, whereas consumption of sucrose, a disaccharide of equal amounts of glucose and fructose does not result in symptoms associated with and observed with fructositis disease. Specifically, research findings indicate that there are separate and distinct pathways for absorption of fructose consumed in excess of glucose as occurs with high dietary fructose and HFCS, versus fructose consumed in equal proportion to glucose as would occur during consumption of sucrose (a disaccharide with digestion products of equal amounts of glucose and fructose). Any excess fructose is absorbed via GLUT5, whereas co-transport of fructose and glucose occurs via GLUT2 (Helliwell, Richardson, Affleck, and Kellett, 2000; Kellett and Helliwell, 2000; Kellett and Brot-Laroche, 2005; Leturque, Brot-Laroche, and Le Gall, 2009; Ramaswamy, Malathi, Caspary, and Crane, 1974).

While the GLUT5 transporter was long believed to be the primary transporter of all forms of fructose (Ushijima, Riby, Fujisawa, and Kretchmer, 1995; Wasserman et al., 1996), more recent studies point to GLUT2 as the primary co-transporter of fructose when consumed in equal proportion to glucose as occurs with the digestion products of sucrose, or as equal amounts of the independent monomers (Helliwell et al., 2000; Kellett and Helliwell, 2000; Kellett and Brot-Laroche, 2005; Leturque et al., 2009; Ramaswamy et al., 1974). The findings that GLUT2 is the co-transporter of equal amounts of glucose and fructose monomers, and of the digestion products of sucrose, whereas GLUT5 is the transporter of excess fructose is significant because it provides a possible explanation and mechanism as to why consumption of high fructose elicits symptoms of fructositis, whereas consumption of sucrose does not. For example, research supports the hypothesis that aberrant or underexpressed GLUT5 may contribute to fructose malabsorption (Helliwell et al., 2000).

The presence of GLUT2 on the apical membrane of enterocytes and its role in fructose transport, was elucidated in a 2000 experiment by Helliwell et al., who demonstrated that its role in fructose transport had eluded researchers for years because GLUT2 is fructose responsive and dynamically transports to the apical membrane via signal transduction involving PKC During previous GLUT2 experiments, its presence was not detected and its role in fructose absorption had remained unknown because PKC is inactivated as soon as experimental intestine is excised resulting in its loss from the brush-border within minutes in vitro (Helliwell et al., 2000). Thus its role in intestinal sugar absorption across the brush-border membrane had remained overlooked until these experiments. These findings are consistent with the previous data described earlier in this text, wherein subjects when challenged with fructose in excess of glucose were found to be malabsorbers as confirmed by the hydrogen breath test and displayed signs of increased gas, and bloating consistent with malabsorption. On the other hand, when subjects were challenged with equal proportions of glucose and fructose, malabsorption results were negative except as observed in children (Riby et al., 1993). The exception to this general rule was seen in young children who experienced malabsorption even with equal proportions of fructose and glucose (Hoekstra et al., 1996).

Despite their apparent similarity, these sweeteners (glucose and fructose chemically bound as sucrose, versus fructose alone, or fructose in higher relative proportion to glucose as occurs with HFCS) are transported differently in the small intestine. This physiological distinction is the key to understanding why consumption of sucrose, a disaccharide of fructose and glucose does not elicit the same adverse reaction as does fructose consumed in excess of glucose. As such, the relatively recent discovery (2000 and 2005) by the aforementioned researchers of the role of GLUT2 transporters in co-transport of fructose with glucose aids in providing insights and answers to the question regarding how elevated fructose and HFCS may trigger immunogens formation, whereas sucrose may not. In GLUT5 deficient individuals, for example, fructose that remains unabsorbed may contribute to elevated intestinal concentrations of fructose, creating conditions of increased reactivity and possible Fru-AGEs formation.

### Understanding how [Present] Fructose Consumption Levels Contribute to Malabsorption

In order to more fully understand the meaning of the fructose malabsorption studies and fructose transport studies it is important to gain a sense of the relative rates of fructose versus glucose consumption in the average American daily diet and to be able to calculate “excess-free-fructose” and the ratio of daily fructose to glucose monomers consumed. To arrive at this calculation it may be relevant to note that in 2011, Ventura et al. found that the relative percentage of fructose in HFCS containing foods they tested was in fact 65%, and not the 55% suggested by producers and the US food industry (Ventura, Davis, and Goran, 2011). According to USDA data, average US per capita consumption of high fructose corn syrup peaked in 1999 at over 1 lb per week and 2010 levels were just under 1 lb per week. Numerous studies cite that a typical U.S. diet includes consumption of 85-100 grams of fructose daily. A simple calculation enables estimation of how much of this consumption is excess-free-fructose. For example, in an HFCS formula of 65% fructose and 35% glucose, if 100 grams represents the 65% component that is fructose, then approximately 54 grams must represent the 35% that is glucose (for a combined fructose and glucose level of 154 grams daily consumption). Excess-free-fructose can then be calculated at approximately 46 grams daily. An analysis using a 55% fructose/45% glucose HFCS formula results in a combined fructose and glucose intake of 182 grams daily consumption, in which 82 grams are glucose and 18 grams are excess-free-fructose (100−82=18 grams excess-free-fructose). A comparison of these numbers to the levels used in the fructose malabsorption research studies enables a better understanding of their results and how to interpret them. For example, at a ratio of 1.8:1 (65% fructose-35% glucose formula) of fructose to glucose consumption and a daily excess-free-fructose consumption level of 46 grams, one might conservatively estimate that 10% to 30% of the average population is likely unable to properly absorb fructose. These are plausible estimates given that researchers found that with a 12 gram serving of fructose, 10% of “healthy adults” displayed symptoms consistent with fructose malabsorption and that with a 2:1 fructose to glucose challenge (50 g to 25 g), 30% of “healthy adults” displayed symptoms of fructose malabsorption. The tables below provide a summary of this data.

Analysis of Excess-Free-Fructose in 100 Grams Daily Consumption of Fructose Primarily from HFCS

| HFCS | | | | |
|---|---|---|---|---|
| formula - | | Total | | |
| fructose/ | Combined | Fructose | Glucose | |
| glucose | grams | grams | grams | Excess-free-fructose |
| 65%/35% | 154 grams | 100 grams | 54 grams | (100 − 54 =) 46 grams |
| 55%/45% | 182 grams | 100 grams | 82 grams | (100 − 82 =) 18 grams |

Percent Testing Positive for Fructose Malabsorption—(Interpreted from Data by Riby et al, 1993; Newman, Gomara et al, 2008)

| | | Percent Test Positive |
|---|---|---|
| | Excess-free-fructose | for fructose malabsorption |
| | component/or | (gas, bloating, |
| Grams challenge | fructose only | positive to breath test) |
| 50 gr glucose/25 | 25 grams excess-free- | 30% (adult) |
| gr fructose | fructose | |
| 12 grams fructose | 12 grams - fructose only | 10% (adult) |
| 15 grams fructose | 15 grams - fructose only | 20% (pediatric) |
| 45 grams fructose | 45 grams - fructose only | 50% (pediatric) |

In a world of unprocessed foods, would we be consuming excess fructose at these levels? In a pre-HFCS world were our ancestors at risk of consuming 46 grams of excess fructose daily? Did they face the same risks of being fructose malabsorbers? An analysis of foods naturally containing fructose and glucose provides the answers to these questions. Most unprocessed, natural foods that are high in fructose are also known to have equivalent amounts of glucose. Only a few foods that might make it onto the daily short list of staples stand out as exceptions. These include apples, pears and watermelons. For example, in the case of an average sized fresh apple, it has approximately 6 grams of fructose for every two grams of glucose which results in a fructose to glucose ratio is more than 2 to 1 (USDA National Nutrient Database for Standard Reference, Release 24. Nutrient Data Laboratory Home Page. 2011). For most other fresh fruits and vegetables the fructose to glucose ratios are slightly below or slightly over 1:1. (This database is accessible online at http://www.nal.usda.gov/fnic/.)

At approximately 4 grams of “excess-free-fructose” per average sized apple, the average consumer would need to eat 8-10 apples per day to consume the 70-100 grams estimate presently being taken in primarily from processed foods mostly in the form of HFCS. How likely is that? Not likely. This means that fructose malabsorption rates are likely high relative to that experienced by generations before us. This is especially true for individuals born after 1980 who have grown up consuming a high fructose diet primarily in the form of the 50-60 dry pounds of HFCS annually.

What can we Learn from Diabetes Research? how does it Relate to Fru-AGE in the GI?

Further insights into the possible mechanisms triggering symptoms of fructositis can be gained from diabetes related AGEs research conducted in the 1980's and by more recent research by Takeuchi et al., 2010. For example, while both glucose and fructose are implicated in AGEs formation, fructose is considered to contribute to AGEs formation more than glucose because the equilibrium for fructose is shifted more to the reactive, open chain form of the molecule relative to glucose (Bunn and Higgins, 1981). It has been estimated that fructose produces ten times more AGEs than does glucose, (Suarez, Rajaram, Oronsky, and Gawinowicz, 1989) despite being observed at much lower concentrations relative to glucose as observed in studies involving diabetic subjects (Takeuchi et al., 2010). The well-studied Maillard reaction when considered in the context of elevated fructose levels in the intestinal tract of fructose malabsorbers/fructose intolerance sufferers, may serve to broaden our understanding of the adverse effects of the biological-AGEs/RAGE/inflammatory axis to include an understanding of its potential role in a dietary-Fru-AGEs/RAGE/asthma/fructositis axis. For example, if a child suffers from fructose malabsorption, by way of underexpressed GLUT5 or some other unknown mechanism, creating a higher than usual concentration of fructose in the small intestine, this condition may increase the probability for fructation (fructosylation) of proteins, peptides, LPs, GPs, and/or amino acids by the Maillard reaction in the small intestines and formation of Fru-AGEs; and subsequent formation of Fru-AGE haptens and Fru-AGE immune complexes within the systemic circulation. This may create conditions wherein byproducts, not normally absorbed via the gut become elevated, get absorbed through the small intestinal epithelium and subsequently absorbed into the blood stream resulting in an immunogenic response. As such, fructated amino acids of such food fragments may become resistant to aminopeptidases normally resident in the small intestine epithelium, wherein the changes in peptide structure and bond character arising from fructation may prevent these proteases (aminopeptidase) from functioning normally during digestion and nutrient absorption.

According to the scientific literature, the Maillard reaction is retarded below pH 6 and accelerated in alkaline conditions (Dills, 1993; Jakas et al., 2008). This is due to the protonation state of the epsilon amino groups of lysines and arginines. At more basic pH, epsilon amino groups become deprotonated, making them better nucleophiles and highly reactive with the exposed carbonyl carbons of the open chain forms of fructose. Conditions in the small intestine (pH 8-9) appear favorable for the reaction, given that large amounts of bicarbonate are introduced to neutralize the chyme deposited in the duodenum from the highly acidic stomach. This is significant because it suggests that the timing for formation of intestinal Fru-AGE is within the “window of time” for digestion, as well as consistent with the proposed hypothesis, and quite different from the timeline observed for AGEs formation in the systemic circulation of diabetic subjects. For example, research conducted to study the kinetics of AGEs formation via the non-enzymatic Maillard reaction in the systemic circulation of diabetics suggests AGEs formation occurs in days to weeks when the carbohydrate moiety is fructose and occurs in weeks to months when the carbohydrate moiety is glucose (Takeuchi, 2010). *This is contrastingly different from the proposed timeline for formation of dietary Fru-AGEs in the duodenum/ileum given the elevated pH 8-9. Dietary Fru-AGEs formation is thought to occur within minutes to hours in the gastro-intestinal tract of those at risk (Robalo-DeChristopher, 2012). The table below with the exception of the last row has been adapted from Fru-AGEs research (Takeuchi et al, 2010).

AGEs Formation—Comparative Reaction Kinetics—Glucose Vs. Fructose at Various pH Levels

| | | | Time to | |
|---|---|---|---|---|
| | | | Intermediates - | Time to Advanced Glycation |
| Sugar Molecule | pH | Time to Schiff Base | Amadori/Heyns | End Products |
| glucose | physiological | hours | days | Weeks to months |
| fructose | physiological | hours | days | Days to weeks |
| *fructose | pH 8-9 | Minutes - *estimated, | Minutes *estimated | Minutes to hours |
| | | exact time not known | exact time not known | *estimated, exact time not known |

*Robalo DeChristopher, 2012

This mechanism gives rise to the aforementioned hypothesis that consumption of elevated fructose and/or HFCS by those at risk, for example young children, fructose malabsorbers, and fructose intolerance sufferers, may lead to formation of fructated (fructosylated), advanced glycation end products (Fru-AGE), in the small intestines and transport through or in between intestinal epithelium. Alternatively, these Fru-AGEs may activate and become taken up by Peyer's Patch M cells (immune cells of the gut). Once across the intestinal barrier, Fru-AGE intestinal antigens may travel mechanistically to the RAGE enriched lungs leading to activation of cells of the immune system including macrophages, dendritic cells, and R and T lymphocytes and may trigger a pro-inflammatory response characterized by release of cytokines and fructositis symptoms including mucus hypersecretion, cough not associated with a cold or flu, fever, inflamed tonsils that if inadequately cleared is associated with chronic bronchitis, allergic rhinitis, ear infections, whistling, wheezing, airway hyper-reactivity and asthma.

As previously cited, recent analysis of the structure of RAGE bound to CEL and CML containing short peptides provided definitive proof that post-translationally modified peptide fragments bind RAGE with much greater affinity than native peptide controls, and that they are capable of oligomerization induced signal transduction resulting in pro-inflammatory cytokines gene expression. The change in charge from positive to negative in post-translationally modified lysines, combined with the extended geometry of the modification is pivotal to the increased binding affinity to the RAGE receptor (Xue et al., 2011).

### Why Tools to Measure AGEs in Diabetes are not Applicable to Fructositis Diagnosis

The methods, antibodies and technologies which exist today for Maillard reaction modified proteins are specific for detection of such AGEs in serum of diabetes patients and offer no value in fructositis related diagnosis. For example, they are used for detection of glycated hemoglobin and glycated albumin useful in diabetes diagnosis. For example, U.S. Pat. No. 4,727,036 issued Feb. 23, 1988 for a monoclonal antibody to HbA1c uses a synthetically derived immunogen comprising the glucosylated N-terminal peptide of HbA1c with 5 to 15 amino acids corresponding to HbA1c that is chemically linked to an immunogenic carrier prior to murine inoculation. In another example, U.S. Pat. No. 5,206,144 issued Apr. 27, 1993, for a monoclonal antibody with specific binding of HbA1c could be used in the determination of HbA1c in the absence of prior denaturation, in blood samples. This patent was later described as having low specificity relative to U.S. Pat. No. 5,646,255 issued Jul. 8, 1997 which utilizes a synthetic formula consisting of three parts including a hapten part which corresponds to the N-terminal end of the HbA1c protein, a spacer and an immunogenic protein. All are used to measure glucose levels in diabetic patients over time.

Such diagnostic tools incorporated herein by reference have no value in detection of AGEs as may be either ingested and transported or in detection of Fru-AGEs formed in the GI and transported in susceptible individuals, for example fructose malabsorbers. Similarly, U.S. Pat. No. 5,702,704 issued Dec. 30, 1997 provides for monoclonal antibodies to the advanced glycosylation endproducts (AGEs) occurring in vivo between circulating proteins with long half-lives and elevated glucose levels that are characteristic of conditions in diabetic patients. Such antibodies are useful in detection of advanced glycation end products complexed with circulating proteins for example, hemoglobin referred to as Hb-AGE, or Albumin-AGE, or collagen-AGE. This tool as described is useful for detecting the onset of either glycemic conditions or diabetes mellitus. In another example is U.S. Pat. No. RE39,138 E issued Jun. 20, 2006 for monoclonal antibody 4G9 derived by immunization of mice with AGE-Keyhole Limpet Hemocyanin (AGE-KLH). The 4G9 antibody is used in sandwich ELISA to detect ApoB-AGE, IgG-Age, and collagen-AGE in serum and to detect their fragments as may be excreted in urine of diabetes patients. All such previous inventions are tools that are useful in the detection of onset of either glycemic conditions or diabetes mellitus, wherein proteins that are resident in serum become glycated due to elevated glucose conditions as occurs in glycemic individuals and those diagnosed with diabetes mellitus. These AGEs related prior inventions are specific to polyclonal and/or monoclonal antibodies for detection and measurement of non-enzymatic glycosylation of normally circulating proteins, and lipoproteins as occurs in glycemic conditions in diabetes. They are of no value in detection of novel and post-translationally modified immunogens as may be either ingested and/or formed via non-enzymatic fructation (fructosylation) of proteins, peptides, lipoproteins, and glycoproteins in the gastrointestinal (GI) tract, or of Fru-AGE-haptens or of Fru-AGE immune complexes as may be formed in the systemic circulation of susceptible individuals, for example fructose malabsorbers.

One of the methods of assessing the presence of disease in which ingested AGEs, intestinally derived Fru-AGEs, systemically derived Fru-AGE-haptens or Fru-AGE-immune complexes are used as biomarkers comprises obtaining a blood or urine sample from a mammal, determining the presence or amount of such AGEs, Fru-AGEs, Fru-AGE-hapten or Fru-AGE immune complexes in the sample and comparing this amount to a standard. With respect to urine samples, peptides and/or protein containing moieties, are excreted in urine at very low levels in normal individuals. As previously described, there are currently available diagnostic tools useful for the detection of AGE-proteins in blood and urine samples, but they are specific for the detection of AGE-proteins arising from glycated human proteins in blood samples and from the turnover of tissue derived AGEs in urine of diabetic patients. They are specific for detection of glycosylated proteins and fragments from for example AGE-albumin, AGE-hemoglobin, and AGE-collagen as occurs in elevated glucose conditions in diabetics. Epitopes from these bio-molecules are very specific and bind diagnostic antibodies that are unique to them. Accordingly, they are not useful in detection or measurement of ingested AGEs, intestinally derived Fru-AGE, Fru-AGE-haptens, nor Fru-AGE-immune complexes. For example, in the case of U.S. Pat. No. 5,646,255, the amino acids valine, histidine, leucine and threonine which are the first four amino acid residues at the n-terminus of the HbA1c protein are bound to the C1 atom of a fructose molecule. Together they form the hapten portion of the hapten-carrier molecule that is used for monoclonal antibody preparation. As such, epitopes used to elicit monoclonal antibody from suitable animals for use in hybridoma production methods are uniquely specific to the amino acid sequences and types of post-translational modifications of the immunogens of said inventions and are of no benefit as relates to dietary AGEs, intestinally derived Fru-AGEs, or those formed in the systemic circulation between these moieties and circulatory proteins such as heat shock proteins, albumin, or hemoglobin—herein referred to as Fru-AGE-haptens, or those formed between these moieties and immunoglobulins herein referred to as Fru-AGE immune complexes.

With respect to specific IgE testing of post-translationally modified antigen as provisioned in U.S. Pat. No. 7,279,295 issued Oct. 9, 2007, the allergen that is the subject of this invention is a post-translationally modified timothy grass pollen with epitope Ph1 p11 that contains glycan structures present on the group 11 allergen. This invention while it pertains to allergy testing to a post-translationally modified antigen, it is for an aero-allergen with a naturally occurring post translational modification that is endogenous to the timothy grass pollen protein. Such tests are not relevant to the diagnostic needs of those with food allergy symptoms that arise from post-translationally modified antigens as may occur in the digestive tract and may complex as hapten or as immune complex in the systemic circulation of those at risk.

The materials and methods of the present invention are also distinguished and different from materials and methods of US patent 2009/011172A1 issued Apr. 30, 2009 which uses a plurality of known and naturally occurring peptides, having allergen epitopes that may be used in immunoassays e.g., micro-array-based immonoassays to predict severity of an allergic response as per US patent number US 2009/0111702. Such allergens as occur naturally within our environment, whether derived by recombinant methods or chemical synthesis, and methods of their detection and quantification, would be of no value in detection, measurement and diagnoses relating to post-translationally modified dietary AGEs, Fru-AGEs, Fru-AGE-hapten or Fru-AGE-immune complexes as may become elevated in fructositis sufferers and as may contribute to other RAGE related disorders including auto-immune diseases, atherosclerosis, heart disease, diabetes and cancer. The same considerations hold true for other patents relating to naturally-occurring-within-the-environment allergens which are considerable in number and too numerable to cite.

The citation of all such patent references herein shall not be construed as an admission that such is prior art to the present invention. Lastly, a search of existing patents failed to surface any patents issued for monoclonal and/or polyclonal antibodies to dietary Fru-AGEs, and/or Fru-AGEs macro-molecules for use as therapeutic agents.

### DESCRIPTION OF THE INVENTION

Therefore the present invention is directed to materials and methods, in particular post-translationally modified peptides, LPs, GPs, and other post-translationally modified food fragments herein referred to as Fru-AGEs; Fru-AGE-haptens; and Fru-AGE-immune complexes for fluorescence enzyme immunoassay (FEIA) and microarray type multiplexed specific immunoglobulin/IgE/IgG testing of serum and bodily fluids; and for monoclonal and polyclonal antibodies production to be used for detection and quantification of ingested AGEs, intestinally derived Fru-AGEs, Fru-AGE-haptens, and Fru-AGE immune complexes in body fluids (for example, urine, serum, and blood) of those at risk, for example fructose malabsorbers, wherein methods may be by antibodies micro-array, ELISA, or other methods. Another application of the present invention comprises use of the monoclonal and polyclonal antibodies as therapeutic agents in the management of associated pro-inflamatory diseases including asthma; COPD; pulmonary disorders; arthritis; rheumatoid arthritis; SLE; Alzheimer's; atherosclerosis; heart disease; peripheral vascular disease (PVD); diabetic retinopathy, neurophathy, and nephropathy; lung cancer; fibrosis; irritable bowel disease; ulcerative colitis; Chron's disease; psoriasis; cancer; and others.

One aspect of the present invention is the provision of a methodology for identification, selection, and design of such target moities suitable for molecular allergy diagnosis by numerous methods as known by those of skill in the art, for example, FEIA, microarray based specific immunoglobulin/IgE/IgG testing and antibody production (polyclonal and monoclonal) useful for ELISA testing in the context of the pro-inflammatory disease states herein previously described. This methodology is relevant given the nature of the variability of the intestinal environment that gives rise to such immunogens, wherein the number of examples possible and within the scope of the present invention is significant. Therefore numerous examples are provided herein of CML and CEL containing Fru-AGEs. These examples are not intended to exclude from the present invention other suitable Fru-AGEs, Fru-AGE-haptens, and Fru-AGE-immune complexes as may be defined utilizing the methodology herein described and within the scope of the present invention.

Included in the methodology of the present invention is the provision of “selection rules” for the identification and selection of all Fru-AGEs containing moieties including the analysis of food proteins, lipoproteins, and glycoproteins in particular those high lysine (K) as may become non-enzymatically modified via the Maillard reaction to contain for example CML or CEL capable of eliciting an immune response and/or RAGE binding. Such analysis includes identification of these residues, for example K, that are flanked by amino acid residues to their left (n-terminus) and right (carboxy-terminus), that once fructated (fructosylated), function as Fru-AGE. For example, only K that are immediately preceded, e.g. flanked to the left, by at least one residue with relatively strong hydrophobic character including for example phenylalanine, isoleucine, tryptophan, leucine, valine, methionine, tyrosine, cysteine, or alanine are selected. As shown by Xue et. al, the hydrophobic surface of RAGE to which these residues bind is large enough to accommodate any (hydrophobic) side chain, possibly providing an increase in binding affinity without a subsequent increase in specificity. With respect to which residues may flank the said K to the right—the rules are far more permissive and may include any residue. For example, it has been shown that fructation disrupts local secondary structure rendering the area immediately around the modified site extremely flexible, suggesting binding by induced fit. This permissiveness is further indicated by the fact that the molecular interactions between RAGE and the Fru-AGE on the CML/CEL carboxy side region are to the Fru-AGE's backbone (versus side chains) and serve to provide stable binding (Xue et. al., 2011). It is possible that some Fru-AGEs may possess multiple such sites, for example targets with more than one hydrophobic residue followed by CML or CEL that are capable of binding RAGE. It is also possible that some Fru-AGEs may possess multiple CML or CEL, but only one or more may have the required left flanking hydrophobic residue critical for RAGE binding, whereas others do not.

Another aspect of the present invention is provision of “selection length rules” for determination of Fru-AGE lengths for use in functional diagnostic tests for example FEIA and specific immunoglobulin/IgE/IgG microarray based assays or for use in animal inoculations for antibodies production. Again, this is useful given the variability of the intestinal environment that gives rise to such immunogens. One possibility is to start the Fru-AGE sequence with the CML/CEL left flanking N-terminus hydrophobic residue as the first residue in the sequence, such that this left flanking residue must satisfy the rules consistent with RAGE binding character—and must be a hydrophobic residue. Subsequent residues will then include all residues to the right of the hydrophobic residue/CEL or CML sequence and continue to achieve a length of at least five, or eight, or fifteen residues, or twenty or twenty five residues, or thirty residues long Fru-AGE sequence. The next possible Fru-AGE shifts the beginning of the sequence one amino acid residue to the left, thereby beginning the sequence at the penultimate to the hydrophobic residue with the effect of dropping one residue off at the right or carboxy-terminus end of the eight or 12, or 15, or 20, or 25 or 30 residues long sequence. This shift to the left may continue for each food derived Fru-AGE by dropping one residue from the right until there is a minimum of four residues to the right (carboxy-side) of the CML/CEL residue.

In certain embodiments the length of the Fru-AGE may be a minimum of 5 or greater, for example, 5, 6, 7, 8, 9, 10 . . . N residues in length, wherein N may be any number that is a subset of the complete protein, or LP, or GP, or Fru-AGE-hapten, or Fru-AGE immune complex or other food molecule. For example, this shift to the left is possible only twice for the eight amino acids long Fru-AGE. The shift to the left may continue in any of the other Fru-AGEs for example the 15, or 20, or 25 or 30 residues long sequences until at minimum four amino acids remain to the right of the CEL/CML residue. The following table illustrates the number of Fru-AGEs possible for a sequence that satisfies the selection rules herein described, and includes examples for the 5, 8, 12 and 15 amino acids long sequences. The table can of course be extended to include 20, 25, 30 or longer Fru-AGEs sequences, and any lengths in between, with continuous shifts to the left, stopping at the point at which a minimum of four residues remain on the right or carboxy side of the hydrophobic/CEL or CML residues. Phenylalanine (F) is used as example of the hydrophobic residue, but as previously stated, can be any one of the hydrophobic residues previously identified including phenylalanine, isoleucine, tryptophan, leucine, valine, methionine, tyrosine, cysteine, or alanine. The CML/CEL immediately to the right of the hydrophobic residue reflects the fructated (fructosylated) residue. It occupies the zero position. The positive numbers reflect right flanking (Carboxy side) residues, and negative numbers reflect left flanking (N-terminus side) residues. Table 1 on the following page provides an illustration of the methodology.

TABLE 1

| Example Number | Sequence illustration | Length |
|---|---|---|
| Example 1 | F (CML/CEL) 1 2 3 | 5 residues |
| Example 2 | F (CML/CEL) 1 2 3 4 5 6 | 8 residues |
| Example 3 | −2 F (CML/CEL) 1 2 3 4 5 | 8 residues |
| Example 4 | −3 −2 F (CML/CEL) 1 2 3 4 | 8 residues |
| Example 5 | F (CML/CEL) 1 2 3 4 5 6 7 8 9 10 | 12 residues |
| Example 6 | −2 F (CML/CEL) 1 2 3 4 5 6 7 8 9 | 12 residues |
| Example 7 | −3 −2 F (CML/CEL) 1 2 3 4 5 6 7 8 | 12 residues |
| Example 8 | −4 −3 −2 F (CML/CEL) 1 2 3 4 5 6 7 | 12 residues |
| Example 9 | −5 −4 −3 −2 F (CML/CEL) 1 2 3 4 5 6 | 12 residues |
| Example 10 | −6 −5 −4 −3 −2 F (CML/CEL) 1 2 3 4 5 | 12 residues |
| Example 11 | −7 −6 −5 −4 −3 −2 F (CML/CEL) 1 2 3 4 | 12 residues |
| Example 12 | −8 −7 −6 −5 −4 −3 −2 F (CML/CEL) 1 2 3 | 12 residues |
| Example 13 | F (CML/CEL) 1 2 3 4 5 6 7 8 9 10 11 12 13 | 15 residues |
| Example 14 | −2 F (CML/CEL) 1 2 3 4 5 6 7 8 9 10 11 12 | 15 residues |
| Example 15 | −3 −2 F (CML/CEL) 1 2 3 4 5 6 7 8 9 10 11 | 15 residues |
| Example 16 | −4 −3 −2 F (CML/CEL) 1 2 3 4 5 6 7 8 9 10 | 15 residues |
| Example 17 | −5 −4 −3 −2 F (CML/CEL) 1 2 3 4 5 6 7 8 9 | 15 residues |
| Example 18 | −6 −5 −4 −3 −2 F (CMU CEL) 1 2 3 4 5 6 7 8 | 15 residues |
| Example 19 | −7 −6 −5 −4 −3 −2 F (CML/CEL) 1 2 3 4 5 6 7 | 15 residues |
| Example 20 | −8 −7 −6 −5 −4 −3 −2 F (CML/CEL) 1 2 3 4 5 6 | 15 residues |
| Example 21 | −9 −8 −7 −6 −5 −4 −3 −2 F (CML/CEL) 1 2 3 4 5 | 15 residues |
| Example 22 | −10 −9 −8 −7 −6 −5 −4 −3 −2 F (CML/CEL) 1 2 3 4 | 15 residues |

Another aspect of the present invention is to utilize well characterized biochemical models of gastroduodenal infant, children and adult digestion that are unique to each cohort in an effort to identify digestion resistant fragments that are more probable for each cohort and thereby good cohort specific fructation (fructosylation) candidates. The infant and young children's model differs from the adult model for example with reduced levels of proteases (for example, eightfold reduced for pepsin and tenfold reduced for trypsin and chymotrypsin), phosphatidylcholine and bile salts (Dupont et al., 2009). Fru-AGEs identified via this method are herein referred to as Model Fru-AGEs for use in immunoassays and animal inoculations for antibodies production that are the subjects of the present invention. Such Model Fru-AGEs may be utilized alone or conjugated with circulatory proteins, for example heat shock proteins to form Fru-AGE-haptens, or complexed for example with immunoglobulins, esRAGE or sRAGE to form Fru-AGE-immune complexes. The following table illustrates examples of several Model Fru-AGEs from cow's milk and cheese including beta-lactoglobulin and beta-casein, and hen's egg white protein, ovalubumin, all selected using the methodology for identification, and selection herein described. This table 2 of examples is not intended to be limiting of all Model Fru-AGEs, or of derivative moieties that contain them (ie. Fru-AGE-haptens, Fru_AGE-immune complexes) and serves only as an example. Other such Model Fru-AGEs and their derivatives may arise from other proteins for example in cow's milk and cheese, eggs, oats, wheat, chicken, red meat, and other food categories.

TABLE 2

| | | CML/CEL containing |
|---|---|---|
| Protein | Species | fragment |
| Beta-lactoglobulin | Bovine | LIVTQTM(CML/CEL)GLDI |
| Beta-lactoglobulin | Bovine | EKT(CML/CEL)IPAVF |
| Beta-lactoglobulin | Bovine | VLDTDY(CML/CEL)KY |
| Beta-lactoglobulin | Bovine | EKFDKAL(CML/CEL)A |
| Beta-lactoglobulin | Bovine | AL(CML/CEL)ALPM |
| Beta-Casein | Bovine | GVSKV(CML/CEL)EAMAPK |
| Ovalbumin | Hen | VLVNAIVF(CML/CEL) |
| | | GLWEKA |
| Ovalbumin | Hen | VYLGA(CML/CEL)DSTR |
| Ovalbumin | Hen | IT(CML/CEL)PNDVYS |
| Ovalbumin | Hen | M(CML/CEL)ILEL |
| Ovalbumin | Hen | I(CML/CEL)VYL |
| Ovalbumin | Hen | ERKI(CML/CEL)VY |
| Ovalbumin | Hen | ESL(CML/CEL)ISQAVHAA |
| | | HAEINE |

Another aspect of the present invention is the provision of kits with Fru-AGEs, Model Fru-AGEs, Fru-AGE-haptens, Fru-AGE-immune complexes, reagents, suitable controls and multi-well plates for use in FEIA or microarray-based immunoassay for example on a microchip. Kits being prepared such that each well or unique chip site contains a unique Fru-AGE, Model Fru-AGE, Fru-AGE-hapten, Fru-AGE-immune complex or suitable control that is individually coating a well within the plate array, or region of the microchip. Assay steps include the use of blocking proteins or buffers designed to block the portions of wells or microarrays that are still reactive after well coating or array printing. This step prevents non-specific binding and eliminates fluorescent background following incubation steps. Well plates or regions of the microchip with Fru-AGEs, Model Fru-AGEs, or their derivatives may be organized by food type for example all milk and cheese, or egg, or chicken, or red meat, or grains, or as may be organized by Fru-AGEs more likely to be observed in young children, or in adults, or as may be more likely observed in specific disease states; or Fru-AGEs, Model Fru-AGEs, Fru-AGE-haptens, and Fru-AGE-immune complex as may be organized by size, or by multiple sizes within the same food type, or as may be organized by targets with sequence homology across food types, for example profilins in fruits, or tropomyosin in meats.

The assay is performed wherein patient samples are introduced into each well plate. Patient's antibodies present within the samples bind to well coating antigens. A washing step clears unbound antibodies from the well. Enzyme labeled antibodies against the bound antibodies are added and incubated. Another washing step follows to clear unbound enzyme labeled antibodies. An incubation step with developing agent follows. Once stopped the signal is measured. The higher the signal, the higher the levels of antibodies present in the sample. The detected signal is proportional to the amount of epitope/immunoglobulin complex at each position in the array. In fluorescence, a type of electromagnetic spectroscopy which analyzes fluorescence from a sample, analysis is conducted using a beam of light, usually UV light that excites the electrons in the molecules of certain compounds causing them to emit light. Fluorescence is then measured via devices known as fluoremeters. In non-fluorescence assays, other types of signals are possible and are measured using other techniques, for example absorption spectroscopy.

Another aspect of the present invention is the use of Fru-AGEs, Model Fru-AGEs, Fru-AGE-haptens, and Fru-AGE-immune complexes that are the subject of the present invention for animal inoculations for production of polyclonal and monoclonal antibodies by conventional, hybridoma or other methods as known by those skilled in the art and/or as herein described for use in detection and measurement of such moieties in bodily fluids of those at risk.

Another aspect of the present invention is the provision of Fru-AGEs and Model Fru-AGEs bound to a carrier protein for example albumin or a heat shock protein or hemoglobin, herein referred to as Fru-AGE-haptens. In one example, the heat shock protein becomes a part of the immunogen. This is distinguished from methods relating to antibodies production wherein a carrier protein serves primarily as adjuvant to elicit an immune response during animal inoculation as herein further described.

Another aspect of the present invention is the provision of monoclonal and polyclonal antibodies as may be utilized for the measurement and quantification of dietary Fru-AGEs, Model Fru-AGEs, Fru-AGE-haptens, and Fru-AGE immune complexes as may be formed via binding to immunoglobulins for example IgG, IgE, or IgA, or as may be formed via binding to endogenous soluble RAGE or soluble RAGE. This comprehensive approach in materials, methods, and design is critical and enables a comprehensive approach to the measurement of dietary derived Fru-AGEs given that previous research demonstrates that quantification and measurement of Fru-AGEs may only be observed within immune complexes (Takeuchi et al., 2010).

Another aspect of the present invention is the provision of antibody kits for use in well characterized sandwich or competitive ELISA or more recently characterized multiple and portable (M & P) ELISA methods as per the following patents: EP 1 499 894 B1 in EPO Bulletin 25.02.209 N. 2009 September; U.S. Pat. No. 7,510,687 in USPTO Bulletin 31.03.2009; ZL 03810029.0 in SIPO PRC Bulletin 08.04.2009; PCT/IT03/00218—WO 03/085401. All such methods being well known to those of skill in the art wherein antibodies are bound to a solid support whether well plates or beads, or by other solid support methods and sample introduced either directly or as in M & P by multi-catcher devices with 8 or 12 immunosorbent ogival pins. The key step in any of these assays is immobilization and detection of antigen in sample fluids. Antigen may be detected directly by antibodies of the present invention that may be designed as labeled primary antibody or indirectly by labeled secondary antibody which is an antibody to the primary antibody. In the sandwich type ELISA, antigens contained within sample fluids are captured between two primary antibodies, for example a monoclonal capture antibody and a polyclonal detection antibody, both being part of the present invention. Fluorescent or other type tags as known by those of skill in the art provide detection and a measure of the amount of antigen in the sample.

In another aspect of the present invention, Fru-AGEs, Model Fru-AGEs, Fru-AGE-haptens and Fru-AGE-immune complexes are synthesized or conjugated with labels or tags using a method as known by those of skill in the art for use in competition assays. In this ELISA variation, unlabeled antigen from samples and the labeled antigen compete for binding to the capture antibody (monoclonal or polyclonal antibody of the present invention) that coats well plates. A decrease in signal from the labeled Fru-AGEs, Model Fru-AGEs, Fru-AGE-haptens, or Fru-AGE-immune complex indicates the presence of such antigens in samples when compared to assay wells with labeled targets only.

The USDA and its research division track and quantify per capita consumption of the principal foods consumed in the United States. Many foods on this list are known to be relatively high in lysines and arginines including milk, cheese, egg, meats, and oats. The Economic Research Service (ERS), the primary source of economic information and research within the U.S. Department of Agriculture estimates 2005 per capita consumption of principal foods in pounds (except as noted) as follows: All dairy products, milk equivalent, milk fat basis—600.5; Flour and cereal products—192.3; Wheat flour—134.1; beef—62.4; chicken—60.4; eggs—256 (in units); potatoes—125.6.

Of this list, the examples used in this invention disclosure as illustrated in Table 3 include proteins in cow's milk and cheese, eggs, oats, wheat, chicken, and potatoes. However, it is not the intention that these examples limit the scope of the invention to this subset. Rather, it is the object of this invention that all ingested AGEs containing foods and intestinally derived post-translationally modified peptides, glycopeptides, lipopeptides, and other food molecules capable of modification by fructation, ie Fru-AGEs, Model Fru-AGEs, Fru-AGE-haptens, and Fru-AGE-immune complex as may be formed within the gastrointestinal tract and/or systemic circulation of those at risk are the subject of said invention, and that this list is not exclusive and further includes all CML and CEL containing food derived peptide, protein, glycoprotein and lipoprotein sequences and isoforms occurring through food processing methods and as may be identified, quantified, measured and derived via the methodology for identification, selection, and design of the present invention.

For preparation, purification, and synthesis of antigen fragments (prior to fructation) food molecules may be derived from natural sources or from any one of a number of well characterized recombinant protein expression methods, or peptide synthesis methods as utilized and known by those skilled in the art. For example, if by recombinant methods, suitable techniques are well established as detailed in many publications including the following, “Production of recombinant proteins: novel microbial and eukaryotic expression systems” by Gerd Gellissen, 2005: ISBN 3527310363 or E. Coli Gene Expression Protocols: Methods in Molecular Biology, Series Volume 205 by Peter E. Vaillancourt, 2011: ISBN13: 9781617373022 and ISBN10: 1617373028.

Or for example, if by chemical synthesis, many protocols including automated methods may be used as known by those of skill in the art and detailed in numerous publications. Examples include but are not limited to the following, Stewart and Young, Solid Phase Peptide Synthesis, 2d ed., Pierce Chemical Co., 1984; Tam et al., J. Am. Chem. Soc., 105:6442, 1983; Fields, Solid-Phase Peptide Synthesis, Academic Press, 1997; Howl, Peptide Synthesis Protocols (Methods in Molecular Biology), Humana Press, 2010; Chan and White, Fmoc Solid Phase Peptide Synthesis: A Practical Approach series, Oxford University Press, 2000. Chemical synthesis methods may include but are not limited to solid phase peptide synthesis, Fmoc/tBu strategy, activation of the carboxyl groups by aminium-derived coupling reagents, use of PEG-modified polystyrene resins, Boc strategy, solution phase synthesis, and HPLC analysis. Many other texts and documents suitable for teaching solid phase synthesis of peptides are available and known to those of skill in the art and may be used to synthesize epitope arrays from any allergen. In certain embodiments, labels are incorporated into the Fru-AGEs, Model Fru-AGEs, Fru-AGE-haptens and Fru-AGE-immune complexes by methods well known by those of skill in the art, of course, the incorporated labels are added such that they do not interfere with binding of targets to capture molecules.

Subsequent to synthesis by recombinant protein expression methods or chemical synthesis, methods of purification and determination of peptide authenticity are pursued as known by those skilled in the art and may include chromatographic purification, purification by mass separation, affinity column, histidine tags/metal binding, immunoaffinity chromatography, HPLC, reverse-phase HPLC, polyacrylamide gel electrophoresis, or amino acid analysis by microwave hydrolysis, or other known methods. If by recombinant protein expression methods, said proteins may need to be subjected to acid and enzymatic digestion steps to derive suitable Fru-AGEs, Model Fru-AGEs, Fru-AGE-haptens, and Fru-AGE-immune-complex.

Once purified, antigen fragments derived from any of the above methods are combined with fructose for the formation of post-translationally modified Fru-AGEs/Model Fru-AGEs, Fru-AGE-haptens, Fru-AGE-immune complex via the non-enzymatic Maillard reaction, a process well known by those skilled in the art, as illustrated in a number of scientific papers including for example by Takeuchi et al., 2010. Such preparation steps include fructation of said peptides, LPs or GPs for modification of target lysines to CML or CEL for the preparation of Fru-AGEs, Model Fru-AGEs, Fru-AGE-haptens and Fru-AGE-immune complex of the present invention. Purification steps are again undertaken to isolate all Fru-AGEs.

In embodiments wherein Fru-AGEs, Model Fru-AGEs, Fru-AGE-haptens, Fru-AGE-immune-complex antibodies are immobilized onto a solid support or substrate, many rigid and semi-rigid materials and methods are possible including membranes, filters, chips, slides, wafers, fibers, magnetic or nonmagnetic beads, gels, tubing, plates, polymers, microparticles, and capillaries. The substrate may have any one of a variety of surface forms including wells, trenches, pins, channels and pores. Immobilization may also be by covalent means via the use of chemical bonding or UV. Examples include Fru-AGEs, Model Fru-AGEs, Fru-AGE-haptens, Fru-AGE-immune complex or antibodies bound to a glass surface that has been modified to contain epoxide or aldehyde groups, or avidin. Other examples include all such molecules transported from solution to a given position on a substrate by electrical means. Solid support binding may also be indirectly through a linker group (of varying lengths) for example polyethylene glycol (PEG), or diamines, or diacids wherein the terminal ends of such linkers interact with reactive groups on the substrate surface and the other end of the linker is designed for binding to molecules of the present invention. In the preferred embodiments, well plates or micro-array of all Fru-AGEs moieties presenting the epitopes are immobilized upon a solid surface, alternatively, it is possible that the epitopes may be in solution.

Since the Fru-AGEs and Model Fru-AGEs capable of binding RAGE and priming T lymphocytes are typically of small size, while they may have the complexity necessary to be antigenic, their small size usually renders them ineffective as immunogens on their own. Such small Fru-AGEs that are to be used as antigens in animal inoculation are conjugated to carrier proteins either directly or through linkers to insure that they induce an immune response and production of antibodies. In preparation for immunization of suitable mammals to obtain polyclonal and subsequently monoclonal antibodies, all Fru-AGEs as necessary may be conjugated with any of a number of well-known carrier proteins including key hole limpet hemocyanin (KLH), Blue Carrier Immunogenic protein, and others. Furthermore, anyone of a number of conjugation methods of hapten-carrier conjugation may be used and are well known by those skilled in the art and include use of chemical moieties for protection of functional groups which are removed in the final steps of processing.

Another aspect of the present invention is to conjugate CML/CEL containing haptens to proteins within the systemic circulation known to form conjugates and complexes with Fru-AGEs, for example heat shock proteins as carrier protein. This approach is consistent with numerous studies which have identified for example Hsp70 and Hsp90 with hapten bound as capable of activating immune cell function (Schmitt et al., 2007; Oura et al., 2012). For example CML/CEL+Hsp may be used as antigens in the form of Fru-AGE-haptens for use in FEIA assays, micro-array based assays,

Another aspect of the present invention is to further conjugate such CML/CEL containing hapten for suitability in animal inoculations, for example as CML/CEL containing hapten+linker+Hsp for polyclonal and monoclonal antibodies production for use in conventional ELISA and high throughput M & P ELISA or other suitable assays.

In the case of monoclonal antibodies, preparation may be for example, by the hybridoma method, or other known recombinant techniques. All immunogens as herein described including Fru-AGEs, or Model Fru-AGEs, or Fru-AGE hapten, or Fru-AGE immune complex, or Fru-AGE macro-molecule or CML/CEL containing hapten+Hsp, or CML/CEL containing hapten+linker+Hsp, or CML/CEL containing hapten+carrier protein, or CML/CEL containing hapten+linker+carrier protein, are added to a suitable buffer solution along with addition of suitable adjuvants. This combination is then injected several times in an appropriate organism and then the antibodies are obtained in a known manner. Examples of appropriate mammals include mice, sheep, rabbits, rats and guinea pigs. Known adjuvants, including for example, Freund's adjuvant are used as nonspecific stimulators of the immune response, helping to deposit or sequester the injected material and causing a dramatic increase in the antibody response. To achieve high antibody titers, the injection is repeated at regular intervals of time, for example, every two to four weeks. At the appropriate time, the animal is bled and polyclonal antibodies obtained in the manner as known by those skilled in the art. With the immunogens derived as per the present invention, there can be obtained highly specific monoclonal antibodies with the use of the well-known hybridization process. One such method is described by G. Kohler and C. Milstein in Science, 208,692 et seq./1980 and Biotechnology. 1992; 24:524-6.

After immunization of the host mammal, B-lymphocytes are isolated from the spleen of the immunized animal, fused with myeloma cells and the hybridoma cells formed are cloned. From the clones formed there are then isolated the cell lines which produce the antibodies which react specifically and with the highest affinity with the target antigen. These clones are cultured to produce the cell line for the desired monoclonal antibody. The antibodies produced by the hybridoma are all of a single specificity and are therefore monoclonal antibodies (in contrast to polyclonal antibodies). The antibody activities are determined in a known manner by those familiar with the art usually via enzyme linked immunoassay, referred to as ELISA. In this example, all immunogens of the present invention used for antibody preparation include those food derived advanced glycation end-products (AGEs, Fru-AGEs, Model Fru-AGEs, Fru-AGE-haptens, Fru-AGE-immune complex) containing N-epsilon-(carboxy-ethyl)lysine, also known as CEL and/or N-epsilon-(carboxy-methyl)lysine, also known as CML, with flanking residues known to bind the RAGE receptor and/or capable of eliciting an immune response. The antibodies obtained as per the procedures herein described are characterized by a high specificity and high affinity to CEL or CML immunogens as might be ingested or formed in the intestinal tract of those at risk, and as may go on to form Fru-AGE-hapten and Fru-AGE-immune complex in the systemic circulation of those at risk.

Another aspect of the present invention is the use of monoclonal and polyclonal antibodies of the present invention to the plurality of Fru-AGEs, Fru-AGE haptens, and Fru-AGE-immune complexes (macro-molecules) for use as therapeutic agents for symptoms management of “fructositis” disease and other [potentially associated] diseases wherein RAGE is elevated, including but not limited to the following: neutrophilic asthma; COPD; pulmonary disorders; arthritis, rheumatoid arthritis; Systemic Lupus Erthematosus (SLE); Alzheimer's; atherosclerosis; heart disease; peripheral vascular disease (PVD); diabetic retinopathy, neurophathy, and nephropathy; lung cancer and fibrosis; irritable bowel disease; ulcerative colitis; Chron's disease; psoriasis; cancer; and others.

As previously stated, the potential for a large number of different dietary AGEs, and intestinally derived Fru-AGEs, Model Fru-AGEs, Fru-AGE-haptens, and Fru-AGE-immune complexes exists. Table 3 provides examples of dietary AGEs, and Fru-AGEs that have been identified using the methodology of the present invention. For each example the species and protein source are identified. The example sequences contain the CML or CEL preceded by the requisite hydrophobic residue or residues as per the identification and selection rules of the present invention. These examples are not intended to be construed as all inclusive but serve simply as examples. They serve as an illustrative subset of the many AGEs, Fru-AGEs, and their derivatives that are within the scope of the present invention. Again, it is not the intention that these examples limit the scope of the invention to this subset. Rather, it is the object of the present invention that all dietary Fru-AGEs and the antibodies that bind them are the subject of said invention including the following: Fru-AGEs, as may arise due to various processing methods; Fru-AGEs as may arise within the GI of those at risk; Fru-AGEs as may arise from dietary and intestinally formed Fru-AGEs forming complexes with proteins of the systemic circulation proteins, including for example Fru-AGE-haptens as previously described herein, and Fru-AGE-immunoglobulin complexes as previously described herein, and Fru-AGE esRAGE and Fru-AGE-sRAGE immune complexes as previously described herein.

Table 3 provides examples of various length AGEs and Fru-AGEs that are amino acid sequences identified from within food proteins and selected for using rules of the present invention. The identified fragments contain the requisite lysines preceded by hydrophobic residues (phenylalanine, isoleucine, tryptophan, leucine, valine, methionine, tyrosine, cysteine, or alanine). These sequences are depicted with the post translational CML/CEL modification. Many more lengths are possible for each Fru-AGE containing fragment, wherein the CML/CEL residue that occupies position zero is flanked to the left or right by varying numbers of residues from within the protein sequence as is illustrated in table 1. By following the fragment design rules of the present invention, fragment lengths may be 5, 6, 7, 8, 9, 10, 11, 12, 13, 14, 15, 16, 17, 18, 19, 20, 21, 22, 23, 24, 25, 26, 27, 28, 29, 30 or longer.

TABLE 3

| | | CML/CEL containing |
|---|---|---|
| Protein | Species | fragment |
| Glutenin, high molecular weight | Triticum aestivum | L(CML/CEL)ACQQVMDQQL |
| subunit DX5 | (wheat) | |
| Glutenin, high molecular weight | Triticum aestivum | LL(CML/CEL)RYYPSVTCP |
| subunit DX5 | (wheat) | |
| Glutenin, high molecular weight | Triticum aestivum | L(CML/CEL)VAKAQQLAAQ |
| | (wheat) | |
| Glutenin, high molecular weight | Triticum aestivum | L(CML/CEL)ACQQVMDQQ |
| | (wheat) | |
| Glutenin, high molecular weight | Triticum aestivum | ALL(CML/CEL)RYYPSVTS |
| | (wheat) | |
| Gamma-gliadin | Triticum aestivum | VL(CML/CEL)TLPTMCNVY |
| | (wheat) | |
| avenin | Avena sativa (oat) | M(CML/CEL)LDSCREYVAE |
| avenin | Avena sativa (oat) | W(CML/CEL)WWKGGCEELR |
| avenin | Avena sativa (oat) | WW(CML/CEL)GGCEELRNE |
| avenin | Avena sativa (oat) | L(CML/CEL)IAKSLPTQST |
| avenin | Avena sativa (oat) | L(CML/CEL)NNRGQESGVF |
| avenin | Avena sativa (oat) | TM(CML/CEL)VVAMQTLPA |
| Seed storage globulin1 | Avena sativa (oat) | FL(CML/CEL)PFVSQQGPV |
| Aspartic protease inhibitor 2 | Solanum turberosum | IATV(CML/CEL)LCVSYTIW |
| | (potato) | (CML/CEL)VGNLNAYF |
| Aspartic protease inhibitor 2 | Solanum turberosum | F(CML/CEL)IV(CML/ |
| | (potato) | CEL)LSNFGYNLLYCPITPPF |
| Aspartic protease inhibitor 7 | Solanum turberosum | V(CML/CEL)LCGSYTIW(CML/ |
| | potato) | CEL)VGNINAHLRTMLLETGG |
| Aspartic protease inhibitor 7 | Solanum turberosum | F(CML/CEL)IV(CML/ |
| | (potato) | CEL)SSKFGYNLLYCPLTRH |
| Aspartic protease inhibitor 11 | Solanum turberosum | V(CML/CEL)LCVSYTIW(CML/ |
| | (potato) | CEL)VGNLNAYFRT |
| Aspartic protease inhibitor 11 | Solanum turberosum | F(CML/CEL)IV(CML/ |
| | (potato) | CEL)LSNFGYNLLYCPITPPFL |
| | | CPFCRDDNFCAKVGVVIQN |
| Aspartic protease inhibitor 9 | Solanum turberosum | IW(CML/ |
| | (potato) | CEL)VGNLNAYFRTMLLETGG |
| | | TIG |
| Aspartic protease inhibitor 9 | Solanum turberosum | F(CML/ |
| | (potato) | CEL)IVKLSNFGYNLLSCPFTS |
| | | IICLRCPEDQFCAK |
| Aspartic protease inhibitor 9 | Solanum turberosum | FKIV(CML/ |
| | (potato) | CEL)LSNFGYNLLSCPFTSIIC |
| | | LRCPEDQFCAK |
| Aspartic protease inhibitor 10 | Solanum turberosum | W(CML/ |
| | (potato) | CEL)VGINAYLRTMLLETGGT |
| | | GQADSSY |
| Aspartic protease inhibitor 10 | Solanum turberosum | F(CML/CEL) |
| | (potato) | IVKSSILGYNLLYCPITRPIL |
| Cysteine protease inhibitor 1 | Solanum turberosum | MTVVYI(CML/ |
| | (potato) | CEL)FFVKTTKL |
| Cysteine protease inhibitor 1 | Solanum turberosum | DQTVW(CML/ |
| | (potato) | CEL)VNDEQLVVT |
| Cysteine protease inhibitor 1 | Solanum turberosum | VGNENDIF(CML/ |
| | (potato) | CEL)IMKTDLV |
| Serine protease inhibitor 7 | Solanum turberosum | YTIW(CML/ |
| | (potato) | CEL)VGDYDASLG |
| Serine protease inhibitor 7 | Solanum turberosum | SWLIV(CML/ |
| | (potato) | CEL)SSQFGYNLL |
| Patatin (potato tuber protein) | Solanum turberosUm | GGGI(CML/CEL)GIIPATILEF |
| | (potato) | |
| Patatin (potato tuber protein) | Solanum turberosum | ISSFDI(CML/CEL)TNKPVIFT |
| | (potato) | |
| Patatin (potato tuber protein) | Solanum turberosum | FASI(CML/ |
| | (potato) | CEL)SLNYKQMLLL |
| Non-specific lipid-transfer | Zea mays (corn) | AACNCL(CML/ |
| protein - zea m14 | | CEL)KNAAAGVSG |
| Actin | Gallus gallus (chicken) | NGSGGLV(CML/CEL)AGFAGDD |
| Actin | Gallus gallus (chicken) | KRGILTL(CML/CEL)YPIEHGI |
| Actin | Gallus gallus (chicken) | DLTDYLM(CML/CEL)ILTERGY |
| Actin | Gallus gallus (chicken) | REIVRDI(CML/CEL)EKLCYVA |
| Actin | Gallus gallus (chicken) | ALAPSTM(CML/CEL)I(CML/ |
| | | CEL)IIAPP |
| Skeletal muscle Tropomyosin | Gallus gallus (chicken) | KKKMQML(CML/ |
| beta | | CEL)LDKENAID |
| Skeletal muscle Tropomyosin | Gallus gallus (chicken) | QGLQKKL(CML/ |
| beta | | CEL)GTEDEV |
| Skeletal muscle Tropomyosin | Gallus gallus (chicken) | EKYSESV(CML/ |
| beta | | CEL)EAQEKL |
| Skeletal muscle Tropomyosin | Gallus gallus (chicken) | DESERGM(CML/ |
| beta | | CEL)VIENRMK |
| Skeletal muscle Tropomyosin | Gallus gallus (chicken) | VIENRAM(CML/ |
| beta | | CEL)DEEKMELQE |
| Skeletal muscle Tropomyosin | Gallus gallus (chicken) | VIENRAM(CML/ |
| beta | | CEL)DEEKME |
| Skeletal muscle Tropomyosin | Gallus gallus (chicken) | MELQEMQL(CML/ |
| beta | | CEL)EAKHI |
| Skeletal muscle Tropomyosin | Gallus gallus (chicken) | GDLEEEL(CML/ |
| beta | | CEL)IVTNNLKSL |
| Skeletal muscle Tropomyosin | Gallus gallus (chicken) | KIVTNNL(CML/ |
| beta | | CEL)SLEAQADKYS |
| Skeletal muscle Tropomyosin | Gallus gallus (chicken) | DKYEEEI(CML/ |
| beta | | CEL)LLGEKL(CML/CEL)EAE |
| Skeletal muscle Tropomyosin | Gallus gallus (chicken) | EVYAQKM(CML/ |
| beta | | CEL)YKAISEELDN |
| Skeletal muscle Tropomyosin | Gallus gallus (chicken) | Y(CML/CEL)AISEELDNAL |
| beta | | |
| Skeletal muscle Tropomyosin | Gallus gallus (chicken) | KKKMQML(CML/ |
| alpha | | CEL)KLDKENAL |
| Skeletal muscle Tropomyosin | Gallus gallus (chicken) | DKYSESL(CML/ |
| alpha | | CEL)KDAQEKLE |
| Skeletal muscle Tropomyosin | Gallus gallus (chicken) | DESERGM(CML/ |
| alpha | | CEL)KVIENRAQ |
| Skeletal muscle Tropomyosin | Gallus gallus (chicken) | EIQEIQL(CML/ |
| alpha | | CEL)KEAKHIAE |
| Skeletal muscle Tropomyosin | Gallus gallus (chicken) | RIMDQTL(CML/ |
| alpha | | CEL)KALMAAED |
| Skeletal muscle Tropomyosin | Gallus gallus (chicken) | DKYEEEI(CML/ |
| alpha | | CEL)KVLTDKLK |
| Skeletal muscle Troponin C | Gallus gallus (chicken) | EEMIAEF(CML/ |
| | | CEL)KAAFDMFDA |
| Skeletal muscle Troponin C | Gallus gallus (chicken) | VMMVRQM(CML/ |
| | | CEL)KEDAKGKS |
| Skeletal muscle Troponin C | Gallus gallus (chicken) | EDIEDLM(CML/ |
| | | CEL)KDSDKNND |
| Skeletal muscle Troponin C | Gallus gallus (chicken) | IDFDEFL(CML/ |
| | | CEL)KMMEGVQ |
| Skeletal muscle Myosin heavy | Gallus gallus (chicken) | HPKESFV(CML/ |
| chain | | CEL)KGTIQSKE |
| Skeletal muscle Myosin heavy | Gallus gallus (chicken) | EGGKVTV(CML/ |
| chain | | CEL)KTEGGETL |
| Skeletal muscle Myosin heavy | Gallus gallus (chicken) | GGETLTV(CML/ |
| chain | | CEL)KEDQVFSM |
| Skeletal muscle Myosin heavy | Gallus gallus (chicken) | PAVLYNL(CML/ |
| chain | | CEL)KERYAAWM |
| Skeletal muscle Myosin heavy | Gallus gallus (chicken) | MHYGNL(CML/ |
| chain | | CEL)FKQKQRE |
| Skeletal muscle Myosin heavy | Gallus gallus (chicken) | MHYGNL(CML/CEL)F(CML/ |
| chain | | CEL)QKQRE |
| Skeletal muscle Myosin heavy | Gallus gallus (chicken) | LNSAELL(CML/ |
| chain | | CEL)ALCYPRV |
| Skeletal muscle Myosin heavy | Gallus gallus (chicken) | ALCYPRV(CML/ |
| chain | | CEL)VGNEFVT |
| Skeletal muscle Myosin heavy | Gallus gallus (chicken) | DEKTAIY(CML/ |
| chain | | CEL)LTGAVMH |
| Skeletal muscle Myosin heavy | Gallus gallus (chicken) | KATDTSF(CML/ |
| chain | | CEL)NKLYDQH |
| Skeletal muscle Myosin heavy | Gallus gallus (chicken) | LYQKSSV(CML/ |
| chain | | CEL)TLALLFA |
| Skeletal muscle Myosin heavy | Gallus gallus (chicken) | RVLYADF(CML/ |
| chain | | CEL)QRYRVLN |
| Skeletal muscle Myosin heavy | Gallus gallus (chicken) | GHTKVFF(CML/ |
| chain | | CEL)AGLLGLL |
| Skeletal muscle Myosin heavy | Gallus gallus (chicken) | VRSFMNV(CML/ |
| chain | | CEL)HWPWMKL |
| Skeletal muscle Myosin heavy | Gallus gallus (chicken) | PWMKLFFKI(CML/ |
| chain | | CEL)PLLKS |
| Skeletal muscle Myosin heavy | Gallus gallus (chicken) | FKIKPLL(CML/ |
| chain | | CEL)SAESEKE |
| Skeletal muscle Myosin heavy | Gallus gallus (chicken) | ERCDQLI(CML/ |
| chain | | CEL)TKIQLEA |
| Skeletal muscle Myosin heavy | Gallus gallus (chicken) | RKLEGDL(CML/ |
| chain | | CEL)LAHDSIM |
| Skeletal muscle Myosin heavy | Gallus gallus (chicken) | MQLQKKI(CML/ |
| chain | | CEL)ELQARIE |
| Skeletal muscle Myosin heavy | Gallus gallus (chicken) | EKEKSEL(CML/ |
| chain | | CEL)MEIDDLA |
| Skeletal muscle Myosin heavy | Gallus gallus (chicken) | RHLEEEI(CML/CEL)A(CML/ |
| chain | | CEL)NALAH |
| Skeletal muscle Myosin heavy | Gallus gallus (chicken) | DKILAEW(CML/ |
| chain | | CEL)QKYEETQ |
| Skeletal muscle Myosin heavy | Gallus gallus (chicken) | SLSTELFKM(CML/ |
| chain | | CEL)NAYEE |
| Skeletal muscle Myosin heavy | Gallus gallus (chicken) | QLELNQI(CML/ |
| chain | | CEL)SEIDRKI |
| Skeletal muscle Myosin heavy | Gallus gallus (chicken) | EEDIDQL(CML/ |
| chain | | CEL)DTQIHLD |
| Skeletal muscle Myosin heavy | Gallus gallus (chicken) | EAEQLAL(CML/ |
| chain | | CEL)GGKKQLQ |
| Skeletal muscle Myosin heavy | Gallus gallus (chicken) | KRSAEAV(CML/ |
| chain | | CEL)GVRKYER |
| Skeletal muscle Myosin heavy | Gallus gallus (chicken) | RKYERRV(CML/ |
| chain | | CEL)ELTYQCE |
| Skeletal muscle Myosin heavy | Gallus gallus (chicken) | LVDKLQM(CML/ |
| chain | | CEL)V(CML/CEL)SYKRQ |

In another embodiment of the present invention, it is expected that new epitopes within known food allergens may arise for example due to processing methods, or due to elevated fructose conditions in the intestines of those at risk, for example fructose malabsorbers. As previously described, allergenicity to such novel epitopes are not possible with the presently available diagnostic tools and methods. As such, the materials and methods of the present invention extends to such post translationally modified novel epitopes (PTMNE) within known food allergens as may occur due to the Maillard reaction resulting in CML and CEL containing PTMNE's capable of binding RAGE. Such list includes but is not limited to the following: Peanut Ara h 1, Ara h 2, Ara h 3, Ara h 4, Ara h 5, Ara h 6, Ara h 7, Ara h 8; brazil nut Ber e 1; European chestnut Cas s 1; Carrot Dau c 4, Dau c.1.0103; Soybean Gly m 2, Gly m 3, Gly m 4, Gly m 1.0101; Barley Hor v.15; English walnut Jun r 1, Jug r 2; Tomato Lyc e 1, Lyc c 2.0102; Apple Mal d 1, Mal d 2, Mal d 3; Banana Mus xp 1; Rice Ory s 1; Avocado Pers a 1; Apricot Pru ar 1, Pru ar 3; Cherry Pru av 1, Pru av 2, Pru av 3, Pru av 4; Plum Pru d 3; Peach Pru p 3; Pear Pyr c 1, Pyr c 4; Sesame Ses 11, Ses 12, Ses 13; White Mustard Sin a 1; Potato Sola T 1, Sola T 2, Sole T 3, Sole T 4; Corn Zea m 14; Kiwi Act c 1; Cashew Ana o 1.0101; Pineapple Ana c 1; Bovine Bos d 4, Bos d 5, Bos d 6, Bos d 8; Chicken Gad d 1, Gad d 2, Gad d 3, Gad d 4, Gad d 5; Crab, Lobster, Shrimp, Scallop, Mussel, Oyster Tropomyosin; Salmon Sal s 1.

1. Peptide containing food fragments of food proteins, lipoproteins, and/or glycoproteins that contain post-translational modifications, for example N-epsilon-(carboxy-ethyl)lysine (CEL) or N-epsilon-(carboxy-methyl)lysine (CML), also known as Fru-AGEs (dietary) for use in immuno-assays.

2. The plurality of fragments of claim 1 that bind the receptor of advanced glycation end products (RAGE), its isoforms, or other immune bio-molecules.

3. The plurality of claim 1 wherein food fragments contain post-translational modifications CEL and/or CML that arise from food processing.

4. The method of claim 1 where fragments are post translationally modified, i.e. fructosylated (fructated) to incorporate modifications (CML/CEL) to their native sequences as may occur in the digestive tract via the well-characterized non-enzymatic Maillard reaction.

5. The plurality of Fru-AGEs from claim 1 are bound to proteins of the systemic circulation as haptens, including for example heat shock proteins, hemoglobin, albumin, or others for use in immuno-assays.

6. The plurality of Fru-AGEs from claim 1 complexed with proteins of the immune system including for example immunoglobulins/IgG/IgE/IgA, endogenous soluble RAGE (esRAGE), soluble RAGE (sRAGE), or others—all herein referred to as Fru-AGE-immuno complexes and/or Fru-AGE-macro-molecules for use in immuno-assays.

7. Monoclonal and polyclonal antibodies to the plurality of Fru-AGEs, Fru-AGE haptens, and Fru-AGE-immune complexes (macro-molecules) from claims 1, 5 and 6 for use in immuno-assays.

8. Monoclonal and polyclonal antibodies to the plurality of Fru-AGEs, Fru-AGE haptens, and Fru-AGE-immune complexes (macro-molecules) from claims 1, 5 and 6 for use as therapeutic agents for symptoms management.

9. The method of claims 1-8 comprising detecting asthma, or neutrophilic asthma, or COPD, or food associated asthma in the subject.

10. The method of claims 1-8 comprising biomarker assessment and disease detection known to be associated with elevated RAGE and inflammation including arthritis, rheumatoid arthritis; systemic lupus Erthematosus (SLE); Alzheimer's; atherosclerosis; heart disease; peripheral vascular disease (PVD); diabetic retinopathy, neurophathy, and nephropathy; lung cancer and fibrosis; irritable bowel disease; ulcerative colitis; Chron's disease; psoriasis; cancer; and others.

11. The method of claims 1-8 comprising use of monoclonal and polyclonal antibodies as therapeutic agents in the management of pro-inflamatory diseases including asthma; COPD; pulmonary disorders; arthritis; rheumatoid arthritis; SLE; Alzheimer's; atherosclerosis; heart disease; peripheral vascular disease (PVD); diabetic retinopathy, neurophathy, and nephropathy; lung cancer; fibrosis; irritable bowel disease; ulcerative colitis; Chron's disease; psoriasis; cancer; and others.

12. Diagnostic kits wherein the plurality of Fru-AGEs, Fru-AGE macro-molecules, and plurality of monoclonal and polyclonal antibodies to the aforementioned are used for well characterized fluorescence enzyme linked immunoassay (FEIA), ELISA, multiple and portable ELISA, micro-array or other immunoassay methods.

13. Diagnostic kits for immunoassay testing wherein bio-molecules of claims 1, 5, 6 and 7 are arrayed on a solid support for example microarray or wells.

14. Diagnostic kits for molecular immunogens, “allergens” diagnosis comprising as reagent—Fru-AGEs, Fru-AGE-haptens, and/or Fru-AGE-immune complexes of claims 1, 5, and 6.

15. Diagnostic kits for molecular immunogens/“allergens” diagnosis comprising as reagent—antibodies of claims 7 and 8.

16. The method of claim 12 wherein the plurality of Fru-AGE (claim 1); Fru-AGE macro-molecules (claims 5 & 6); monoclonal and polyclonal antibodies (claim 7); anti-immunoglobulin/anti-IgE/anti-IgG/anti-IgA; (e) sRAGE, anti-(e)sRAGE—are labeled and/or enzyme linked.

17. The method of claims 7 and 8 wherein Fru-AGEs of claims 1, 5 and 6 are used for animal inoculations for production of polyclonal and monoclonal antibodies by conventional, hybridoma or other methods.

18. The method of claim 5 is distinguished from methods relating to antibodies production wherein a carrier protein serves primarily as adjuvant to elicit an immune response during animal inoculation.

19. The method of claims 1, 5, and 6 where fragments are identified, selected and lengths chosen such that lysines and/or arginines are preceded by a hydrophobic residue for example phenylalanine, isoleucine, tryptophan, leucine, valine, methionine, tyrosine, cysteine, or alanine.
a) The method of claims 1, 5, and 6 wherein the fragment contains a minimum of four residues to the right of the post-translationally modified lysine, and/or arginine.
b) The method of claims 1, 5, and 6 wherein fragments are a minimum of 5 amino acids in length and may extend to any length, for example, 5, 6, 7, 8, 9, 10 . . . N residues, wherein N may be any number that is a subset of the complete protein, or LP, or GP.
c) The method of claims 1, 5 and 6 wherein amino acid position one of the fragment begins with the CML/CEL left flanking N-terminus hydrophobic residue is the first residue in the sequence.
d) The method of claims 1, 5 and 6 wherein the next possible Fru-AGE shifts the beginning of the sequence one amino acid residue to the left, thereby beginning the sequence at the penultimate to the hydrophobic residue with the effect of dropping one residue off at the right or carboxy-terminus end of the previous fragment to arrive at a new fragment length.
e) The method of claims 1, 5 and 6 wherein shifts to the left may continue for each dietary Fru-AGE by dropping one residue from the right until there is a minimum of four residues to the right (carboxy-side) of the CML/CEL residue.
f) A method of claims 1, 5 and 6 wherein fragments are arrayed on a solid support as a series wherein the amino acid sequence of the next fragment in the series shifts by one amino acid to the left as per claims 15 a-e.

20. The method of claims 1, 5, 6 where a model digestion system is used to identify amino acid sequences and lengths of peptide containing food fragments.
a) The method of claim 20 wherein the model digestion system is one that mimics the biochemical environment of the digestive tract of different individuals.
b) The method of claim 20 wherein the biochemical environments reflect that of different age groups including for example young children, adolescents, and adults.
c) The method of claim 20 wherein the biochemical environments reflect that of individuals with different microbiome profiles, or with different disease diagnoses/profiles.
d) The method of claim 20 wherein adjustments are made to pH and/or to concentrations of proteases, phosphatidylcholine and bile salts for the purpose of “digesting” food proteins, lipoproteins, and glycoproteins into “model” peptide containing fragments for fructosylation into Fru-AGEs.

17. The method of claims 1, 5, 6, 7 and 8 that includes the use of blocking proteins or buffers designed to block the portions of wells or microarrays that are still reactive after well coating or array printing to prevent non-specific binding.

18. The method of claims 1, 5, 6, and 7 wherein antigens and/or antibodies are organized and arrayed in groups, for example by food type; or by sequence homology across food types, for example profilins in fruits, or tropomyosin in meats; or organized and arrayed as may be more likely observed in young children, or in adults, or as may be more likely observed in specific disease states.

19. The method of claims 1, 5 and 6 which comprises contacting wells or micro-arrays coated with a plurality of Fru-AGE antigens and/or Fru-AGE macro-molecules with serum obtained from a test subject and determining the amount of serum immunoglobulin (antibodies) or esRAGE or sRAGE bound using labeled anti-antibodies and anti-RAGE antibodies for example anti-IgE, anti-IgG, anti-sRAGE, anti-esRAGE.
a) The method of claims 1, 5 and 6 wherein patient samples are introduced into each well plate.
b) The method of claims 1, 5 and 6 wherein patient's antibodies present within the samples bind to well coating antigens.
c) The method of claims 1, 5 and 6 wherein a washing step clears unbound antibodies from the well.
d) The method of claims 1, 5 and 6 wherein labeled/enzyme labeled antibodies against the bound antibodies are added and incubated and another washing step follows to clear unbound enzyme labeled antibodies.
e) The method of claims 1, 5 and 6 wherein an incubation step with developing agent follows.
f) The method of claims 1, 5 and 6 wherein after incubation the signal is measured. The higher the signal, the higher the levels of antibodies present in the sample and the detected signal is proportional to the amount of epitope/immunoglobulin complex at each position in the array or wells.
g) The method of claims 1, 5 and 6, wherein in fluorescence, a type of electromagnetic spectroscopy which analyzes fluorescence from a sample, analysis is conducted using a beam of light, usually UV light that excites the electrons in the molecules of certain compounds causing them to emit light and fluorescence is then measured via devices known as fluoremeters.
h) The method of claims 1, 5 and 6 wherein, in non-fluorescence assays, other types of signals are possible and are measured using other techniques, for example absorption spectroscopy.

20. The method of claims 1, 5, 6, and 7 wherein signal levels from immunoassays can be used as bio-markers of disease.

21. The method of claims 1, 5, 6 and 7 wherein each well or unique chip site contains a unique Fru-AGE, Fru-AGE macro-molecule, polyclonal antibody, monoclonal antibody, (e) sRAGE, or suitable control that is individually coating a well within the plate array, or region of the microchip.

22. Diagnostic kits for antibody immunoassay wherein monoclonal and/or polyclonal antibodies of claim 7 are arrayed on a solid support for example wells or microarrays for fluids detection of Fru-AGEs and/or Fru-AGEs macro-molecules.
a) The method of claim 7 wherein antibodies are organized and arrayed by reactivity to immunogens by food type; or by sequence homology across food types, for example profilins in fruits, or tropomyosin in meats; or as may be observed in different age groups, for example children, adolescents, or adults; or as may be observed in specific disease states.
b) The method of claim 7 which comprises contacting wells or micro-arrays coated with the plurality of monoclonal and/or polyclonal antibodies with serum obtained from a test subject and determining the amount of serum Fru-AGEs and Fru-AGEs-immune complexes bound using labeled antibodies as per well characterized antibody microarray.

23. Another aspect of the present invention is the provision of antibody kits for use in well characterized sandwich or competitive ELISA or more recently characterized multiple and portable (M & P) ELISA methods. wherein antibodies are bound to a solid support whether well plates or beads, or by other solid support methods and sample introduced either directly or as in M & P by multi-catcher devices with 8 or 12 immunosorbent ogival pins.
a.a) The key step in any of these assays is immobilization and detection of antigen in sample fluids. Antigen may be detected directly by antibodies of the present invention that may be designed as labeled primary antibody or indirectly by labeled secondary antibody which is an antibody to the primary antibody.
a.b) In the sandwich type ELISA, antigens contained within sample fluids are captured between two primary antibodies, for example a monoclonal capture antibody and a polyclonal detection antibody, both being part of the present invention.
a.c) Fluorescent or other type tags as known by those of skill in the art provide detection and a measure of the amount of antigen in the sample.

24. A method of claims 1, 5, 6 and 7 wherein Fru-AGEs, Model Fru-AGEs, Fru-AGE-haptens and Fru-AGE-immune complexes are synthesized or conjugated with labels or tags using a method as known by those of skill in the art for use in competition assays.
a) In this ELISA variation, unlabeled antigen from samples and the labeled antigen compete for binding to the capture antibody (monoclonal or polyclonal antibody of the present invention) that coats well plates. A decrease in signal from the labeled Fru-AGEs, Model Fru-AGEs, Fru-AGE-haptens, or Fru-AGE-immune complex indicates the presence of such antigens in samples when compared to assay wells with labeled targets only.

25. A method of claims 1, 5 and 6 wherein peptide containing fragments may be derived from natural sources.

26. A method of claims 1, 5 and 6 wherein peptide containing fragments may be obtained from any one of a number of well characterized recombinant protein expression methods, or peptide synthesis methods as utilized and known by those skilled in the art.

27. A method of claims 1, 5 and 6 wherein peptide containing fragments may be obtained by chemical synthesis, using many protocols including automated methods as known by those of skill in the art.

28. A method of claims 1, 5, 6, 7 and 8 where in certain embodiments, labels are incorporated into the Fru-AGEs, Model Fru-AGEs, Fru-AGE-haptens, Fru-AGE-immune complexes, monoclonal and polyclonal antibodies by methods well known by those of skill in the art.

29. The methods of claims 1, 5, 6, 7 and 8 for purification and determination of molecule authenticity. Methods may include chromatographic purification, purification by mass separation, affinity column, histidine tags/metal binding, immunoaffinity chromatography, HPLC, reverse-phase HPLC, polyacrylamide gel electrophoresis, or amino acid analysis by microwave hydrolysis, or other known methods. If by recombinant protein expression methods, said proteins may need to be subjected to acid and enzymatic digestion steps.

30. The method of claims 1, 5, 6, and 7 wherein immobilization onto a solid support or substrate may utilize many rigid and semi-rigid materials and methods including membranes, filters, chips, slides, wafers, fibers, magnetic or nonmagnetic beads, gels, tubing, plates, polymers, microparticles, and capillaries. The substrate may have any one of a variety of surface forms including wells, trenches, pins, channels and pores.

31. The method of claims 1, 5, 6 and 7 wherein immobilization may also be by covalent means via the use of chemical bonding or UV or bound to a glass surface that has been modified to contain epoxide or aldehyde groups, or avidin. Other examples include all such molecules transported from solution to a given position on a substrate by electrical means.

32. The method of claims 1, 5, 6 wherein antigens, and of claim 7 wherein antibodies are immobilized onto a solid support binding indirectly through a linker group (of varying lengths) for example polyethylene glycol (PEG), or diamines, or diacids wherein the terminal ends of such linkers interact with reactive groups on the substrate surface and the other end of the linker is designed for binding to molecules of the present invention.

33. The method of claims 1, 5, 6 and 7 where it is possible that the epitopes may be in solution.

34. The method of claims 7 and 8 wherein monoclonal/ and or polyclonal antibodies preparation may be by the hybridoma method, or other known recombinant techniques including use of suitable adjuvants and animals inoculations of for example mice, sheep, rabbits, rats and guinea pigs.

35. The method of claims 7 and 8 wherein small Fru-AGEs that are to be used as antigens in animal inoculation are conjugated to carrier proteins either directly or through linkers to insure that they induce an immune response and production of antibodies. Well known carrier proteins may include key hole limpet hemocyanin (KLH), Blue Carrier Immunogenic protein, and others.

36. The method of claims 5, 7 and 8 wherein anyone of a number of conjugation methods of hapten-carrier conjugation are used including use of chemical moieties for protection of functional groups which are removed in the final steps of processing.
